(19) United States
(12) Patent Application Publication
(10) Pub. No.: US 2005/0282283 A1
(43) Pub. Date: Dec. 22, 2005

(54) Electroporation apparatus and methods

(76) Inventors: Laura Vozza-Brown, Carlsbad, CA (US); Harry Yim, Vista, CA (US); John Cameron, Glasgow (GB); Matthew O'Banion, Vista, CA (US); Adam Scott Henry, Oceanside, CA (US); Lisa Marie Olivier, San Diego, CA (US)

Correspondence Address: FISH & RICHARDSON P.C., PO BOX 1022, MINNEAPOLIS, MN, 55440-1022, US

(21) Appl. No.: 11/109,297

(22) Filed: Apr. 19, 2005

Related U.S. Application Data
(60) Provisional application No. 60/563,709, filed on Apr. 19, 2004.

(51) Int. Cl. A01H 1/00; C12N 15/82

(52) U.S. Cl. 435/459

(57) ABSTRACT

This document discloses electroporation vessels, electrocompetent cells that have been aliquoted and frozen in electroporation vessels, and a number of other apparatuses, kits, and methods for electroporation. Some embodiments of electroporation vessels described herein may include a pair of opposing walls that are downwardly angled toward one another in a gap between two electrode surfaces. Further embodiments of devices and methods described herein may eliminate the need for an end user to transfer competent cells from a capped tube to electroporation cuvette, thereby saving time and producing less waste.

### CROSS-REFERENCE TO RELATED APPLICATION

This document claims priority to U.S. Provisional Application Ser. No. 60/563,709, filed on Apr. 19, 2004 by Vozza-Brown et al., entitled COMPETENT CELLS AND ELECTROPORATION VESSELS, the contents of which are incorporated herein by reference.

### TECHNICAL FIELD

This document relates to methods, systems, and materials for electroporation.

### BACKGROUND

Cloning operations in the biotechnology field often involve “transforming” a host cell with an exogenous nucleic acid, such that the nucleic acid is introduced and maintained (transiently or stably) in the host. Methods for rendering a host cell capable of taking up and maintaining exogenous nucleic acids by transformation (i.e., making them “transformable” or “competent”), and for transforming them are well known and can be practiced as a matter or routine by those skilled in the art.

One relatively efficient transformation method is “electroporation.” Electroporation involves generating transient pores in host cell membranes by exposing the cells to brief electrical impulses. Through the process, permeability of the cells is induced, material is moved into the cells from their surrounding medium, and viable cells are recovered. Nucleic acids can enter the host cells via the transient pores. Small charged molecules, and other large molecules can also be used, such as plasmids, antisense oligonucleotides, RNA, and proteins. The electrical impulses are generally delivered to the suspension of cells by a pair of electrodes located on each side of the suspension.

Cells suitable for electroporation (“electrocompetent cells”) typically are suspended in a hypotonic medium lacking significant amounts of conductive salts. Electrocompetent cells can be obtained from commercial sources; typically frozen and packaged in capped plastic tubes. In electroporation, exogenous nucleic acid is added to the electrocompetent cells and the mixture is exposed to brief electrical pulses using an “electroporator” device that makes electrical pulses suitable for electroporation. Afterward, the cells are allowed to divide and make new cells that contain copies of the exogenous nucleic acid.

### SUMMARY

This document discloses electroporation vessels, electrocompetent cells that have been aliquoted and frozen in electroporation vessels, and a number of other apparatuses, kits, and methods for electroporation. Some embodiments of electroporation vessels described herein may include a pair of opposing walls that are downwardly angled toward one another in a gap between two electrode surfaces. In such embodiments, the electroporation vessel may provide efficient access to material in the gap between the electrode surfaces and may reduce the likelihood of air pocket formation. Further embodiments described herein may eliminate the need for an end user to transfer competent cells from a capped tube to electroporation cuvette, thereby saving time and producing less waste.

In some embodiments, an electroporation vessel may include first and second electrode surfaces that are disposed substantially parallel to one another. The first and second electrode surfaces may be separated by a gap space. The electroporation vessel may also include a cavity to contain electrocompetent cells. At least a portion of the cavity may be disposed in the gap space between the first and second electrode surfaces. The electroporation vessel further includes frozen electrocompetent cells disposed in the cavity.

In one aspect, the portion of the cavity that is disposed in the gap space between the first and second electrode surfaces is an electroporation well. The electroporation well may include a pair of opposing walls that are downwardly angled toward one another in the gap space. In such circumstances, the electroporation well may include a substantially V-shaped portion. Also, the electroporation well may include a pair of substantially vertical walls that join with the pair of downwardly angled walls.

In another aspect, the portion of the cavity disposed in the gap space between the first and second electrode surfaces may be configured to accommodate a liquid volume of about 35 μL to about 220 μL.

In yet another aspect, the vessel may further include a cap member to cover an opening of the cavity. The cap member may comprise a tab extending in a generally upward direction.

In one aspect, the electrocompetent cells may be maintained in the cavity at a temperature of about −20° C. to about −120° C.

In a further aspect, the electrocompetent cells disposed in the cavity may be selected from a number of types. For example, the electrocompetent cells may be selected from the group consisting of prokaryotic, yeast, insect, mammalian, rodent, hamster, primate, human, bird, and plant cells. In some embodiments, the electrocompetent cells may be selected from the group consisting of Escherichia sp., Klebsiella sp., Streptomyces sp., Streptocococcus sp., Shigella sp., Staphylococcus sp., Erwinia sp., Bacillus sp., Serratia sp., Pseudomonas sp., Salmonella sp., Aspergillus sp., Candida sp., Colletotrichum sp., Cryptococcus sp., Dictyostelium sp., Pichia sp., Saccharomyces sp., Schizosaccharomyces sp., algal, maize, tobacco, wheat, rice, Arabidopsis, sheep, cow, horse, and goat cells. In some embodiments, the electrocompetent cells may be selected from the group consisting of E. coli, B. cereus, B. subtilis, B. megaterium, P. aeruginosa, P. syringae, S. typhi, S. typhimurium, P. pastoris, S. cerevisiae, human, monkey, mouse, rat, hamster, and chicken cells. In some embodiments, the electrocompetent cells may be cells of E. coli strains K, B, C, or W, for example, DH10B cells or DH10BT1 cells.

In certain embodiments, an electroporation vessel may include first and second electrode surfaces that are disposed substantially parallel to one another. The first and second electrode surfaces may be separated by a gap space. The electroporation vessel may also include a well to contain electrocompetent cells. The well may comprise a pair of opposing walls that are downwardly angled toward one another in the gap space between the first and second electrode surfaces.

In one aspect, the well comprises a substantially V-shaped portion.

In another aspect, the electroporation vessel may further include a pair of opposing intermediate walls disposed above the well. The opposing intermediate walls may be downwardly angled toward one another along a direction that is substantially perpendicular to a direction of slope of the downwardly angled walls in the well. In a further aspect, the well may be configured to accommodate a liquid volume of about 35 μL to about 220 μL.

In yet another aspect, the gap space is operable to receive a tip portion of a pipette.

In one aspect, the electroporation vessel may further include a body portion having a substantially quadratic shape, a top portion having a substantially circular cross-sectional shape, and an upper cavity disposed in the top portion and the body portion. In such cases, at least one aperture may be formed in a bottom side of the body portion so as to provide access to a bottom electrode surface. Also, the body portion may have rounded corners so as to fit within an electroporator device.

In another aspect, the vessel may further include a cap member having a tab extending in a generally upward direction.

In some embodiments, an electroporation vessel may include first and second electrode means. The first and second electrode means may be disposed substantially parallel to one another and may be separated by a gap space. The electroporation vessel may also include a tapered means for containing electrocompetent cells in the gap space.

In one aspect, the tapered containing means may comprise a pair of guide means. The guide means may be disposed opposite another and may be downwardly angled toward one another in the gap space between the first and second electrode means. In such circumstances, the pair of guide means comprises a pair of walls that are downwardly angled toward one another such that the tapered means comprises a substantially V-shaped portion in the gap space.

In another aspect, the electroporation vessel may further include a body means for retaining the first and second electrode means. The body means may comprise means for electrically accessing the first and second electrodes through a bottom side of the body means.

In some embodiments, a method of preparing electrocompetent cells for electroporation includes adding electrocompetent cells to an electroporation vessel. The vessel may comprise one or more electrodes, an upper cavity, and an electroporation well in communication with the upper cavity. The electrocompetent cells may be added to the electroporation well. The method also includes freezing the electrocompetent cells in the electroporation well.

In one aspect, the method further may include adding a cover over the cavity. In such circumstances, the cover may include a tab extending in a generally upward direction.

In another aspect, the method may further include packaging the electroporation vessel for shipment in an arrangement configured to maintain the cells in a substantially frozen state.

In a further aspect, the cells may be frozen at a temperature ranging from about −20° C. to about −120° C. For example, the cells may be frozen at a temperature of about −90° C. In addition, the method may further include storing the vessel at a temperature ranging from about −20° C. to about −120° C.

In one aspect, the electroporation well in which the electrocompetent cells are added may comprise a pair of opposing walls that are downwardly angled toward one another in a gap space between two electrode surfaces. In such circumstances, the electroporation well may be configured to accommodate a liquid volume of about 35 μL to about 220 μL. Also in such circumstances, the electroporation well in which the electrocompetent cells are disposed may include a substantially V-shaped portion in the gap space.

In another aspect, a portion of the cavity may be disposed above the electrodes may be configured to accommodate a liquid volume of about 700 μL to about 1,100 μL.

In a further aspect, the electrocompetent cells disposed in the well may be selected from a number of types. For example, the electrocompetent cells may be selected from the group consisting of prokaryotic, yeast, insect, mammalian, rodent, hamster, primate, human, bird, and plant cells. In some embodiments, the electrocompetent cells may be selected from the group consisting of Escherichia sp., Klebsiella sp., Streptomyces sp., Streptocococcus sp., Shigella sp., Staphylococcus sp., Erwinia sp., Bacillus sp., Serratia sp., Pseudomonas sp., Salmonella sp., Aspergillus sp., Candida sp., Colletotrichum sp., Cryptococcus sp., Dictyostelium sp., Pichia sp., Saccharomyces sp., Schizosaccharomyces sp., algal, maize, tobacco, wheat, rice, Arabidopsis, sheep, cow, horse, and goat cells. In some embodiments, the electrocompetent cells may be selected from the group consisting of E. coli, B. cereus, B. subtilis, B. megaterium, P. aeruginosa, P. syringae, S. typhi, S. typhimurium, P. pastoris, S. cerevisiae, human, monkey, mouse, rat, hamster, and chicken cells. In some embodiments, the electrocompetent cells may be cells of E. coli strains K, B, C, or W, for example, DH10B cells or DH10BT1 cells.

In certain embodiments, a method for electroporating cells includes thawing frozen electrocompetent cells while the cells are in an electroporation vessel having one or more electrodes. The method may also include electroporating the electrocompetent cells.

In one aspect, at least a portion of the frozen electrocompetent cells may be thawed while in contact with the one or more electrodes of the electroporation vessel.

In another aspect, the frozen electrocompetent cells may be thawed while being disposed in an electroporation well of the electroporation vessel, the electroporation well comprising a pair of opposing walls that are downwardly angled toward one another in a gap space between two electrode surfaces. Also in such circumstances, the electroporation well in which the electrocompetent cells are disposed may include a substantially V-shaped portion in the gap space.

In some aspects, the electrocompetent cells are electroporated with one or more nucleic acid molecules. In such circumstances, the operation of electroporating the electrocompetent cells may cause at least a portion of the electrocompetent cells to become transformed cells. Also in such circumstances, the method may further include removing the electroporated cells from the electroporation vessel, and transferring the electroporated cells to a cell proliferation medium.

In one aspect, the electroporation vessel may comprise a substantially quadratic body portion with rounded corners to fit within an electroporator device that applies the electric field.

In another aspect, the electric field may be applied by accessing the bottom surfaces of the one or more electrode and electrically contacting the bottom surfaces of the one or more electrodes.

In yet another aspect, the method may further include covering an opening of the electroporation vessel with a cap member when electroporating the electrocompetent cells. In such circumstances, the cap member may have a tab extending in a generally upward direction.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of portions of a cuvette in accordance with some embodiments.

FIG. 2 is a cross-sectional top view of the cuvette of FIG. 1.

FIG. 3 is a perspective cross-sectional view of a cuvette in accordance with some embodiments.

FIG. 4 is a perspective view of a cap for a cuvette in accordance with some embodiments.

FIG. 5 is another perspective view of the cap of FIG. 4.

FIG. 6 is a perspective cross-sectional view of a cuvette in accordance with some embodiments.

FIG. 7 is a perspective view of a cuvette in accordance with some embodiments.

FIG. 8 is a perspective exploded view of another embodiment of a cuvette.

FIG. 9 is a perspective cross-sectional view of the cuvette of FIG. 8.

FIG. 10 is a cross-sectional front view of the cuvette of FIG. 8.

FIG. 11 is a perspective view of the cuvette of FIG. 8.

FIG. 12 is a side view of the cuvette of FIG. 8.

FIG. 13 is a front view of the cuvette of FIG. 8.

FIG. 14 is a bottom view of the cuvette of FIG. 8.

FIG. 15 is a top view of the cuvette of FIG. 8.

FIG. 16 is a perspective cross-sectional view of a cuvette in accordance with some embodiments.

FIG. 17 is a perspective view of a cover for a cuvette in accordance with some embodiments.

FIG. 18 is another perspective view of the cover of FIG. 17.

FIG. 19 shows a chart in accordance with an illustrative example.

FIG. 20 shows another chart in accordance with an illustrative example.

FIG. 21 shows another chart in accordance with an illustrative example.

FIG. 22 shows another chart in accordance with an illustrative example.

FIG. 23 shows another chart in accordance with an illustrative example.

Like reference symbols in the various drawings indicate like elements.

### DETAILED DESCRIPTION OF ILLUSTRATIVE EMBODIMENTS

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As referred to herein, electrocompetent cells refers to cells having the ability to take up and establish an exogenous nucleic acid molecule upon electroporation.

As used to herein, nucleic acid molecule refers to any nucleic acid molecule that can be used to transform an organism. Such nucleic acid molecules can include DNA molecules or RNA molecules including antisense RNA, of any size, from any source, including DNA from viral, prokaryotic, and eukaryotic organisms. A nucleic acid molecule can be in any form, including, but not limited to, linear or circular, and single or double stranded. Non-limiting examples of DNA molecules include plasmids, vectors, and expression vectors.

As used to herein, a cloning vector refers to a plasmid, phage DNA, a cosmid, or other DNA molecule which can replicate autonomously in a host cell, and which can be characterized by one or a small number of cloning sites which can be cut in a determinable fashion without loss of an essential biological function of the vector, and into which a nucleic acid molecule such as an oligonucleotide can be spliced in order to bring about its replication and cloning. Exemplary cloning sites in a vector include restriction endonuclease sites. Other exemplary sites include recombination cloning sites, as are know in the art (e.g., Gateway vectors; Invitrogen, Carlsbad, Calif.; see, e.g., U.S. Pat. Nos. 5,888,732, 6,143,557, 6,171,861, 6,270,969, and 6,277,608). The cloning vector can further contain a marker suitable for use in the identification of cells transformed with the cloning vector. Exemplary markers include those that provide resistance to transformed cells, such as tetracycline resistance or ampicillin resistance.

As used herein, expression vector refers to a vector similar to a cloning vector but which can be used to express one or more genes contained in the vector, after transformation into a host. The cloned gene is usually placed under the control of (i.e., operably linked to) control sequences such as promoter sequences.

As used herein, an electroporation vessel refers to any vessel, such as an electroporation cuvette, that can contain electrocompetent cells and can be used in performing electroporation methods on the electrocompetent cells.

As used herein, cells stable for storage refers to the condition of electrocompetent cells that are able to withstand storage for extended periods of time at a suitable temperature, without appreciably losing their transformation efficiency and/or viability. By the term “without appreciably losing their transformation efficiency and/or viability” is meant that the cells maintain sufficient transformation efficiency to yield a minimum number of transformants upon electroporation. Typically, electroporation can yield as many as about 2×10⁶ transformants. Accordingly, sufficient transformation efficiency refers to the ability to yield about 40% to 100%, about 60% to 100%, about 70% to 100%, or about 80% to 100% transformation efficiency. For example, cells can be stored for any amount of time that permits a yield of about 1×10⁶ transformants, or more. Some storage periods are 30 days, 60 days, 90 days, or 120 days, at a temperature of −20° C. or less. Suitable storage temperatures for the cells can vary from below about 0° C. to about −180° C. The storage temperature may range from about −20° C. to about −120° C., about −20° C. to about −90° C., such as −80° C. The storage period or time can range from about 0 days to about 180 days (e.g., 6 months), or longer.

Provided herein are materials and methods for electroporation, and methods for preparing such materials. Also provided herein are electroporation vessels suitable for storing frozen electrocompetent cells and methods of using such vessels. Also provided herein are electroporation vessels in which can be stored frozen electrocompetent cells and methods of using such vessels. Also provided herein are electroporation vessels containing frozen electrocompetent cells, methods of making such vessels and methods of using such vessels. Such vessels can be brought to a temperature at which the electrocompetent cells can thaw and be ready for nucleic acid molecule addition and electroporation.

Conventional electroporation protocols typically require frozen electrocompetent cells to be thawed and transferred to an electroporation cuvette. Exogenous nucleic acid molecule then is added, and the mixture is exposed to brief electrical pulses using an “electroporator” device that makes electrical pulses suitable for electroporation. The transfer step from capped tube to electroporation cuvette is generally inefficient and time-consuming. The apparatuses and methods provided herein can be used to decrease the number of steps required in performing electroporation, which permits electroporation to be performed in less time, and with fewer cell manipulation steps. Because electrocompetent cells can be sensitive to manipulation steps, reducing the number of manipulation steps may increase the efficiency of transformation of electrocompetent cells.

A. Embodiments of Electroporation Vessels

The apparatus provided herein for storage of frozen electrocompetent cells and electroporation of cells can have any of a variety of configurations known in the art. An electroporation vessel may include a cavity into which a suspension of electrocompetent cells can be placed, and two electrodes adjacent to the cavity that can contact the electrocompetent cell suspension placed into the cavity. Any of a variety of electroporation vessels can be used in the methods and apparatuses provided herein, including quickly replaceable electrode assemblies and vessels that can draw in or expel cell suspensions, as exemplified in U.S. Pat. No. 5,422,272; cuvettes that accommodate inserts with porous membranes, as exemplified in U.S. Pat. No. 6,713,292; and vessels that can be coupled with a pump or other means for introducing samples into the vessel and withdrawing samples from the vessel, as exemplified in U.S. Pat. No. 5,676,646. Some useful embodiments of electroporation vessels are described in the figures and description provided herein.

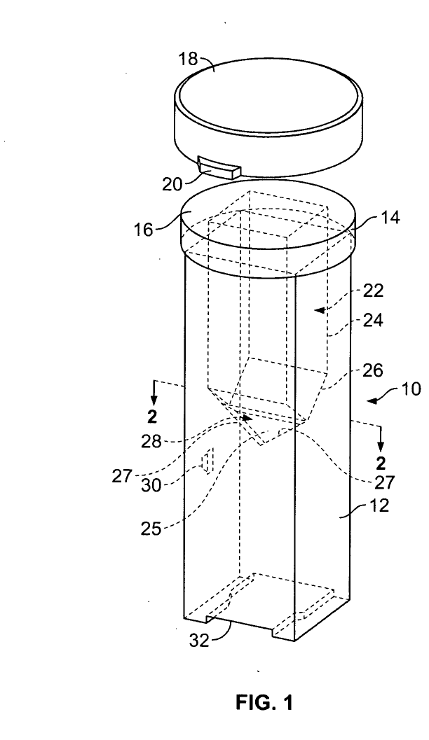

FIG. 1 shows a perspective view of an electroporation vessel in the form of cuvette 10, and is provided to exemplify one of a variety of possible embodiments of the electroporation vessel contemplated herein. The cuvette 10 can have a body 12 and a top portion 14. The body 12 can be any of a variety of shapes, including circular, oval, rectangular or square in cross-section. As depicted in FIG. 1, this embodiment of the cuvette 10 has a substantially square cross-sectional shape and has exemplary dimensions of approximately 12.5 mm by 12.5 mm. A vessel can have a top portion 14, which can be any of a variety of shapes including oval, rectangular, square or circular (as depicted in FIG. 1), and can have a cavity 22 of any of a variety of shapes (shown as a quadratic passage depicted in dashed lines) through its interior portion. A vessel can optionally include a sealing layer 16, which can act as to cover top portion 14, and can be adhered to top portion 14 by any of a variety of methods known in the art. Sealing layer 16 can be formed of any of a variety of materials that form a seal, such as foil or plastic materials.

In some embodiments, a cover 18 can be provided on cuvette 10 in conjunction with, or as an alternative to, sealing layer 16, where cover 18 can protect sealing layer 16, and/or prevent biological materials from exiting cuvette 10. The cover 18 can be provided with a tab 20 or other appropriate mechanism to assist a user in removing the cover 18 conveniently. On its bottom side (not shown in FIG. 1), the cover 18 can have a flat inner surface that rests atop the sealing layer 16, and an inward-facing peripheral lip at its lower edge to hook on the bottom edge of top portion 14. The cover 18 can be made out of an appropriate flexible material, so that it can be snapped on or off of cuvette 10 and does not unintentionally separate from cuvette 10. Any of a variety of materials can be used for cover 18, including, but not limited to, low density polyethylene.

A vessel can also optionally contain an inset 32 on the bottom surface of body 12. This inset 32 can provide for proper seating and/or orientation of cuvette 10 when it is placed in an electroporator, since the inset can mate with a corresponding extension of the electroporator. As shown in FIG. 1, the inset 32 is stepped, with a wider portion at the rear of the body 12 and a narrower portion at the front. The inset 32 could take any number of other shapes that aid in proper seating and/or orientation of the cuvette 10. For example, the inset could be tapered from back-to-front, or could be a shallow cylindrical cut-out in the center of the bottom edge of the body 12. One or more optional projections 30 also can be provided on the side of the body 12 to orient and/or stabilize the cuvette 10 in an electroporator. A projection 30 can be any shape or size to be accommodated by the electroporator in order to orient and/or stabilize the cuvette 10. As shown in FIG. 1, the projection 30 is a narrow arc near the middle of one side of body 12. The projection 30 serves to orient and/or stabilize the cuvette 10 in an electroporator, where the electroporator has a slot that corresponds to the projection 30. As an alternative to or in addition to one or more projections 30, one or more indentations can be provided on the side of the body 12 to orient and/or stabilize the cuvette 10.

Cuvette 10 defines a cavity 22 on its inner portion that is accessible from the top of cuvette 10 through top portion 14. Cavity 22 can be any of a variety of shapes and sizes, having any of a variety of cross section shapes such as oval, circular, rectangular or square. As depicted in FIG. 1, the cavity 22 is generally quadratic in cross-section, extending downward into the body 12.

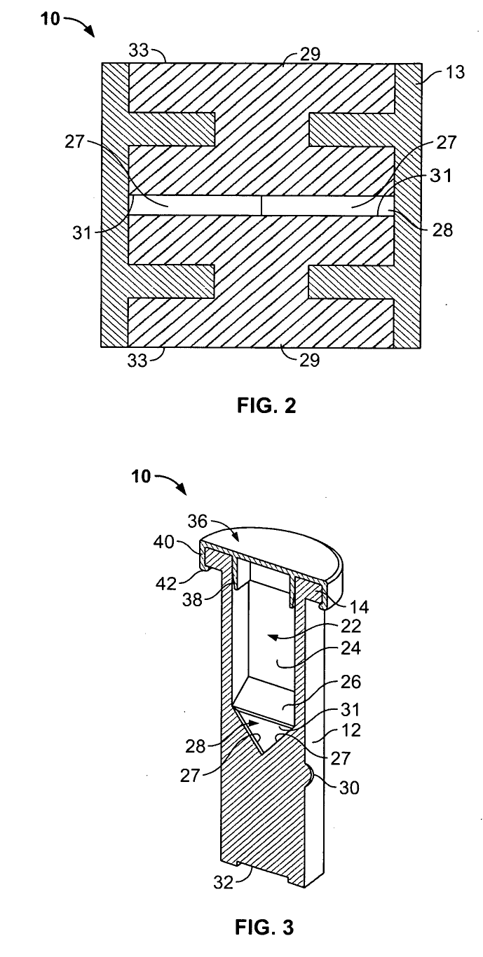

The lowermost portion of cavity 22 may be tapered. For example, cavity 22 can taper downwardly to form a point or a well, such as well 28 of FIG. 1. The tapering at the bottom of cavity 22 can take any of a variety of shapes, including, but not limited to, downward conical, downward pyramidal, or downward V-shaped wedge. In some embodiments, the lowermost portion of cavity 22 is at least partially defined by two parallel walls, such as two parallel walls of opposing electrodes (removed from the view in FIG. 1 for purposes of better showing the well 28; refer to FIGS. 2-3 for an illustrative example of the electrodes).

In downwardly tapering, cavity 22 can take any of a variety of shapes. In one example, the top of cavity 22 can have a quadratically shaped cross-section, and the quadratic shape can be narrowed to form a rectangularly shaped cross-section by one or more walls of cavity 22 downwardly angling inward. An example of such narrowing is seen in FIG. 1, where side wall 24 of cavity 22 meets angled wall 26 at the bottom edge of side wall 24. In FIG. 1, a symmetric angled wall is provided on the facing side of cavity 22. This tapered region of cavity 22 can then lead to a further tapered region such as well 28 of FIG. 1, and thereby serve as an intermediate tapered region. The intermediate tapered region need not be the region where cells reside in performing electroporesis methods; instead this tapered region can serve to narrow the cavity and direct the cells to the region where cells reside in performing electroporesis methods. Thus, presence of an intermediate tapered region is optionally present, and is not required in all embodiments.

In FIG. 1, angled wall 26 is shown as a flat plane, but could take a variety of other forms. For example, angled wall 26 could be curved, angled wall 26 could have a flat portion and a curved portion, or angled wall 26 could meet with other angled walls along its edges. The wall on the facing side can be angled or not angled, and if angled, can, but is not required to be symmetric to angled wall 26. In addition, all four side walls 24 of cavity 22 can meet four angled walls 26, which are similarly shaped or not, which are symmetric or not. Thus, cavity 22 can be configured such that it contains one or more angled walls, according to the shape of cavity 22 and the tapering of cavity 22 to be accomplished. For example, the bottom of cavity 22 could be shaped as an inverted pyramid or as an inverted cone. Angled wall 26 can continue until it meets symmetric angled wall 26 on the facing side of cavity 22 to form the bottom of cavity 22, or angled wall 26 can lead to well 28, which forms the bottom of cavity 22. In such embodiments, bottom of cavity 22 is the location at which the suspension of electrocompetent cells can be placed for performing electroporation.

At the lowermost portion of cavity 22 can be a well 28 into which electrocompetent cells can be placed. FIG. 1 depicts an exemplary well, where angled walls 26 of cavity 22 lead to a V-shaped well 28 at the bottom of cavity 22. In FIG. 1, well 28 is at least partially defined by two facing and parallel V-shaped walls 25 at the bottom of cavity 22. As described in more detail below, these opposing parallel walls 25 may comprise portions of opposing surfaces 31 of two electrodes 29 that are separated by a gap space (e.g., portions of the electrode surfaces 31 are exposed to the contents in the well 28). Also, the well 28 may be at least partially defined by two opposing walls 27 that are downwardly angled toward one another. In such embodiments, the well 28 tapers to a point or rounded vertex and forms a V-shape well that is oriented substantially perpendicular to the V-shape intermediate tapered region (formed by symmetrically angled walls 26). In FIG. 1, the two walls 25 of well 28 are parallel, and can be spaced any of a variety of distances apart. The electrodes 29 (not shown in FIG. 1) may be located at the parallel walls 25 of well 28 so that portions of opposing electrode surfaces 31 (see, for example, FIG. 3) at least partially define the well 28. The spacing is selected to provide for appropriate operation in an electroporator, according to factors known in the art, including, but not limited to, cell type to be electroporated, conditions of the cell suspension buffer, and power and time length to apply in electroporation. An electroporator will apply a voltage or potential differential across the two electrodes 29 on each of the two parallel walls 25 of well 28.

A downwardly tapering shape, such as the V-shaped bottom of the well 28 and the V-shaped intermediate tapered region of the cavity 22, can provide several advantages. For example, the V-shape can make it easier for a user to access materials in the well 28 and the cavity 22. In particular, if the bottoms of the well 28 and the intermediate cavity region were squared-off, a user would have to reach into all of the corners to obtain the material, but the V-shape funnels the material to one lowest point where it can then be removed. The V-shape also guides the point of any removal instrument to the bottom of the well 28, where the material will be. The V-shape may also help prevent air from becoming lodged in the cuvette. Trapped air can be undesirable because it can have a different conductivity than the cell suspension in the cuvette 10, and can thereby induce electrical arcing across the well 28.

In some embodiments, well 28 can be shallow. In other embodiments described in more detail below, a cuvette may include a deepened well. FIG. 1 shows an exemplary shallow well 28, which extends less than substantially all of the way to the bottom of the cuvette 10. The angled walls 27 that form the bottom of well 28 can be angled such that the desired volume can be accommodated in well 28, while the bottom of well 28 remains relatively close to the top of well 28. Exemplary dimensions for well 28 are provided elsewhere herein. The walls 27 that define well 28 need not be flat, and can take any of a variety of shapes, including curved shapes. In some circumstances, the shallow configuration for well 28 allows for easier access to the cells of a pipette or other vesicle used to add exogenous material such as nucleic acid molecules, to the cells. Also in certain cases, the shallow configuration for well 28 also allows for easier commingling of exogenous material and cells in suspension in well 28. For example, a cell suspension can be placed in well 28, and then nucleic acid molecule or other materials to be incorporated into the cells can be provided at the top of well 28 or in well 28. Cuvette 10 or the cell suspension in well 28 can then be agitated to mix the exogenous material into the cellular suspension.

Provided herein is an exemplary cuvette configuration, where certain variations will be apparent to one skilled in the art. The length of the cuvette 10, from the bottom of body 12 to the upper end of top portion 14 can be about 45.3 mm. The body 12 can be approximately quadratic in cross-section, with a dimension of about 12.2 mm by 11.9 mm. The cavity can also be quadratic, with a dimension of 9.4 mm. The side wall 24 of cavity 22 can be about 19 mm long, and the angled wall 26 can be 4.0 mm long.

The well 28 can be described by any of a number of dimensions, including thickness of the well, width of the well and depth of the well, or volume of the well, or the cross sectional area to depth ratio of the well, or the ratio of the longer of the width or thickness relative to the depth of the well. The following description applies to dimensions of the cuvette depicted in FIGS. 1-3, and one skilled in the art can readily apply this description to any of a variety of other electroporesis vessel configurations.

The thickness of the well may represent the dimension of the well that separates the two electrodes (e.g., the gap space between the exemplary electrodes 29 in FIG. 2). The thickness can be any of a range of sizes according to the volume of sample to be accommodated in the well and according to the intended separation of the electrodes. In one embodiment, the thickness of the well is selected according to the intended separation of the electrodes. For example, the thickness can range from about 0.05 cm to about 0.5 cm, from about 0.08 cm to about 0.4 cm, from about 0.1 cm to about 0.4 cm, from about 0.2 cm to about 0.3 cm. Exemplary thicknesses are about 0.1 cm, about 0.2 cm, or about 0.4 cm.

The width of the well can be any size that can be formed within an electroporation vessel such as an electroporation cuvette and also serves to accommodate the intended sample volume and, optionally, that has the intended size relative to the well depth. The well width may be downwardly tapered, such that the width is largest at the top of the well and smallest at the bottom of the well. In one example, when the cuvette has a quadratic shape with dimension of 12.5 mm, the width at the top of the well can be from about 10 mm to about 8 mm, and can taper to a point at the bottom of the well. In one embodiment, as the thickness of the well increases, the width can be decreased while still accommodating the intended volume. In embodiments in which the well is shallow, the width of the well at any particular thickness will be no less than a size required to accommodate an intended volume of sample without requiring an unintendedly large well depth. In one example, the width at the top of the well can be equal to or greater than the well depth. In other examples, the width to depth ratio can be 1:1.5, 1:1.0, 1:0.7, 1:0.5, 1:0.4, 1:0.3, or 1:0.2. In another example, the width can be of a size such that the horizontal cross sectional area at the top of the well (width×thickness) is at least about 0.03 cm², at least about 0.05 cm², at least about 0.08 cm², at least about 0.1 cm², at least about 0.12 cm², at least about 0.15 cm², at least about 0.2 cm², at least about 0.3 cm², or at least about 0.4 cm².

The depth of the well can be any size that can be formed within an electroporation vessel such as an electroporation cuvette and also serves to accommodate the intended sample volume and, optionally, that has the intended size relative to the well width. If the thickness of the well is increased for a particular embodiment, the depth can be decreased while still accommodating the intended volume. In embodiments in which the well is shallow, the depth of the well at any particular thickness will be no less than a size required to accommodate an intended volume of sample without requiring an unintendedly large well depth. In one example, the depth can be equal to or less than the well width. In another example, the depth can be of a size such that the ratio of the horizontal cross sectional area at the top of the well (width×thickness) to the depth is at least about 1:20 (e.g., 0.1 cm² in area: 2 cm in depth), at least about 1:15 (e.g., 0.1 cm² in area:1.5 cm in depth), at least about 1:10 (e.g., 0.1 cm² in area:1 cm in depth), at least about 1:7 (e.g., 0.1 cm² in area:0.7 cm in depth), at least about 1:5 (e.g., 0.1 cm² in area:0.5 cm in depth), at least about 1:3 (e.g., 0.1 cm² in area:0.3 cm in depth), at least about 1:2 (e.g., 0.1 cm² in area:0.2 cm in depth), or more, including 1:1. In one example, the bottom walls 27 of the well can be straight (i.e., not curved), and slope downwardly at angles varying from 30-60 degrees, such as 40-50 degrees, including 45 degrees, relative to a line perpendicular to the long axis of the cuvette.

In another embodiment, the well dimensions are determined according to the volume of liquid the well 28 can accommodate. An exemplary well can accommodate a 35 μL liquid sample. Well volume can vary as a function of the thickness of the well. In some examples, a well 28 with thickness of 0.1 cm can accommodate a volume of liquid up to about 100 μL, up to about 90 μL, up to about 80 μL, up to about 70 μL, up to about 60 μL, up to about 50 μL, up to about 45 μL, up to about 40 μL, up to about 35 μL, up to about 30 μL, or up to about 25 μL. In one embodiment, well 28 can be configured such that when accommodating sample, such as, for example, a 35 μL sample, the surface area to volume ratio of the sample is about 50 m⁻¹ or greater, 100 m⁻¹ or greater, 150 m⁻¹ or greater, 200 m⁻¹ or greater, 250 m⁻¹ or greater, 300 m⁻¹ or greater, 400 m⁻¹ or greater, or 500 m⁻¹ or greater. In further examples, a well 28 with thickness of 0.2 cm can accommodate a volume of liquid up to about 200 μL, up to about 180 μL, up to about 160 μL, up to about 140 μL, up to about 120 μL, up to about 100 μL, up to about 90 μL, up to about 80 μL, up to about 70 μL, up to about 60 μL, or up to about 50 μL. In some examples, a well with thickness of 0.4 cm can accommodate a volume of liquid up to about 400 μL, up to about 360 μL, up to about 320 μL, up to about 280 μL, up to about 240 μL, up to about 200 μL, up to about 180 μL, up to about 160 μL, up to about 140 μL, up to about 120 μL, or up to about 100 μL.

The electrodes formed in the electroporation vessel can be configured in any of a variety of manners. The electrodes may include a first portion that can contact a solution in the electroporation vessel, such as a cell suspension in the electroporation vessel, and the electrodes may also include a second portion that can connect to a power source. FIG. 2 provides an exemplary electrode configuration, showing a cross-section looking down into the well 28 of the cuvette 10 in FIG. 1. The well 28 is bounded on two sides by electrodes 29, which in the figure take an “H-shaped” form. One arm of the “H” serves as a flat contact area 31 in the well 28, and the other arm serves as a flat contact area 33 on the outer surface of the cuvette. The cross-bar of the “H” serves to pass electrical energy from the outside of the cuvette 10 to the well 28. Housing 13 largely surrounds the electrodes 29, and extends into the areas between the arms of the “H”, so that the housing 13 holds the electrodes 29 tightly in place and helps maintain a proper spacing between the opposed facing surfaces 31 of the electrodes 29. The electrodes 29 can extend vertically in the cuvette 10 from near the top edge of the well 28 to near the bottom edge of the cuvette 10. The electrodes 29 can extend less than the full length of the cuvette 10, for example, when the well 28 is shallow, electrodes 29 could be made to be about the same length as the well depth. However, the electrodes can also be made full length, so that they can be used with cuvettes with a variety of well depths ranging from full-depth wells to shallow wells. In addition, some full-length electrodes also allow for more contact area outside the cuvette with electroporator device. The electrodes 29 can be manufactured from metallic or electrically conductive materials, including aluminum, stainless steel, copper, gold, or silver. For example, the electrodes 29 may comprise a 6063-T6 aluminum alloy. In some cases, the electrodes may be finished by plating with gold, silver, copper, or zinc.

The electrodes can also take other appropriate forms for delivering electrical energy in the cuvette. For example, the electrodes can simply be thin metal films placed in the cuvette or can be produced by metal deposition or selective etching, such as by methods disclosed in Gise and Blanchard, Modern Semiconductor Fabrication Technology, Ch. 8 (Prentice-Hall, England Cliffs, N.J.) (1986). The layers can be deposited on a mold into which plastic or other suitable material is introduced to produce the remainder of cuvette 10. See Printed Circuit Handbook, Ch. 1 and 8 (Coombs, Jr., ed.; McGraw-Hill, NY, N.Y.) (1976). The electrodes then can adhere to the plastic or other material, and can be removed from the mold with the rest of cuvette 10. The electrodes can likewise be produced and located according to any other appropriate method.

The electrodes can be spaced apart by any of a variety of distances suitable for the cells to be electroporated. Suitable spacings of electrodes for different cells are known in the art and are readily available from any of a variety of sources. For example, the gap space (e.g., the well thickness) can range from about 0.08 cm to about 0.5 cm, from about 0.1 cm to about 0.4 cm, from about 0.2 cm to about 0.3 cm, or from about 0.22 cm to about 0.28 cm. Exemplary spacings between electrodes for particular cells include about 0.1 cm to about 0.4 cm for insect cells, about 0.2 cm for yeast cells, about 0.2 cm for algae, and about 0.2 to about 0.4 cm for various eukaryotic cells. As referred to herein, “about” as applied to spacings of electrodes may refer to a spacing of a tolerance of 15% or less. Thus, a spacing of about 0.1 cm can range from 0.115 cm to 0.085 cm. In some embodiments, the tolerance can be 10% or less. Some manufacturing processes may provide a gap spacing between the electrodes with a tolerance of 30% or less.

FIG. 3 is a perspective view in partial cross-section of an electroporation cuvette 10 having a sealing cover 36. Cuvette 10 is the same cuvette 10 as that shown in FIG. 1, with body 12, top portion 14, projection 30, and inset 32. A cavity 22 is defined in body 12 in part by side wall 24 and angled wall 26. A well 28 is formed at the bottom of cavity 22. Unlike the cuvette in FIG. 1, the cuvette of FIG. 3 has no foil layer covering the top portion 14. Instead, cover 36 serves to seal the cuvette 10. In particular, a descending portion 38 is arranged to fit in a sealing arrangement with the inside surfaces of the walls 24 that define cavity 22, and thus to act as a stopper for material in cuvette 10. The cover 36 can be further sealed and held in place by a circumferential descending band 40 that is terminated in an inward-facing circumferential lip 42 that can hook on a bottom edge of top portion 14. This combination holds cover 36 tight, but allows it to be snapped on or off cuvette 10 without unnecessary problems. The descending portion 38 can also be tapered inward slightly to aid in the insertion and/or removal of cover 36. Although the descending portion 38 is shown as hollow so that its walls flex and permit for a tighter seal and simplified removal, the descending portion 38 could also be solid or otherwise enforced.

FIG. 4 is a perspective view of the top of a cover 36 for an electroporation cuvette. As shown, cover 36 is provided with an extension 37, in the form of a smoothly integrated arc. Extension 37 is configured to extend outward from the side of the cuvette, and thereby be easily caught by a user's thumb or finger for removing the cover 36 from a cuvette. While cover 36 is shown as round in cross-section to match the round top portion of a cuvette, it could also take any other appropriate shape.

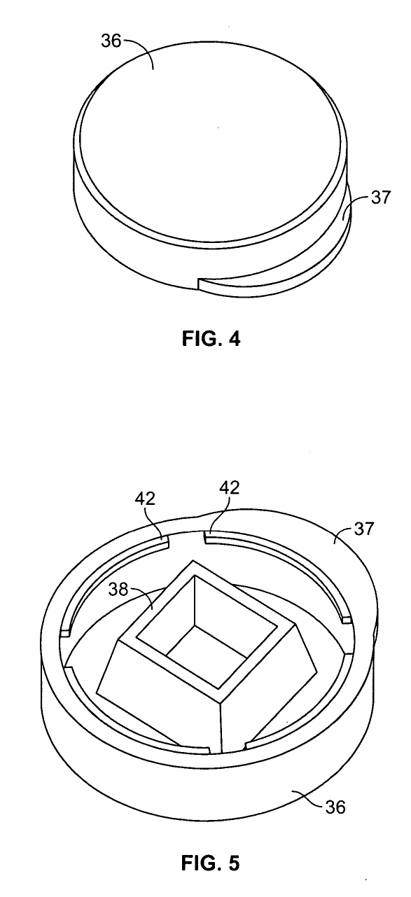

FIG. 5 is a perspective view of the bottom of a cover 36 for an electroporation cuvette. As shown, extension 37 extends outward from cover 36 to provide for easier removal of cover 36. Inward-facing circumferential lip 42 also is provided to hook on a shoulder on the cuvette, and to thereby hold cover 36 more tightly on the cuvette. Inward-facing circumferential lip 42 can be continuous or can have multiple extensions separated by gaps. The inward-facing circumferential lip 42 can have one end higher than the other, and directed toward the top of cover 36 from one end to the other (like threads). In this manner, the cover 36 can be placed more easily on and/or removed more easily from a cuvette, and can allow for twisting of the cover 36 onto and/or off of a cuvette. Also, gaps between inward-facing circumferential lip 42 portions can allow air to escape as the cover 36 is being placed on and/or being removed from a cuvette, and thereby provide for easier mounting and/or removal of the cover 36, and allow for a better seal between cap 36 and the cuvette. In some embodiments, the exterior of a top portion of the cuvette can also be provided with threads, whether formed as threads that extend from top portions, or that are formed cut into the top portion. The cap 36 can then be provided with corresponding threads to allow for a screw-on cap type of cover 36. Alternatively, the cover 36 can be smaller than the upper portion of the cuvette, and can be provided as a plug that fits into the cavity of the cuvette. The plug can be provided with a shoulder portion that is larger than the cavity of the cuvette, and thereby prevents the plug from falling into the cavity.

Still referring to FIG. 5, descending portion 38 is configured to correspond to the inside shape and size of the cuvette cavity. Descending portion 38 can be molded as a single piece with the rest of cover 36, or could be attached to, or otherwise molded into, cover 36. The other walls of descending portion 38 can taper inward slightly from the top of cover 36 to the bottom, so that cover 36 can be inserted onto a cuvette more easily. The thickness of the walls of descending portion 38 can be selected so that the walls are sufficiently flexible to allow for insertion into a cuvette, yet sufficiently strong to press hard against the cuvette and thereby form a tight seal.

A variety of cuvette variations are contemplated in the methods, apparatuses and compositions provided herein. Exemplary variations include variations of well volume and depth, and variations of electrode configuration.

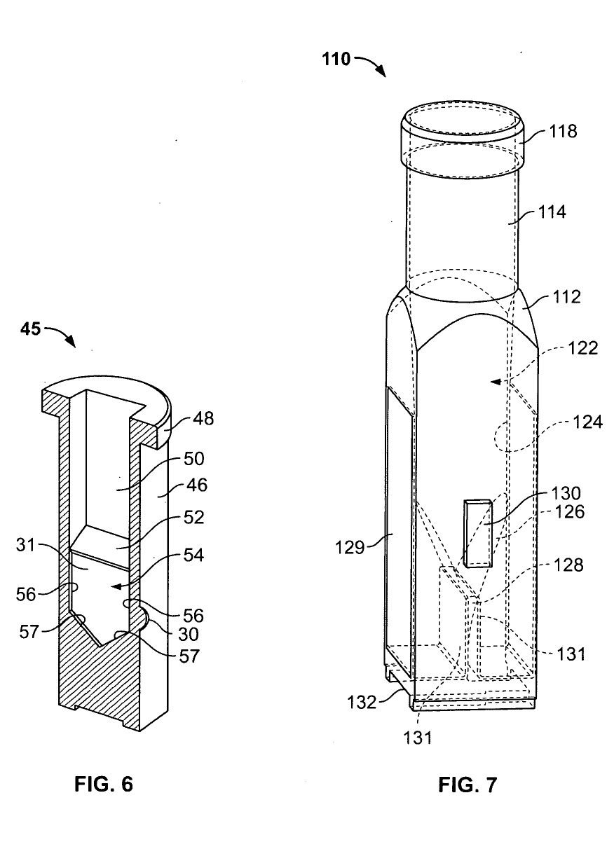

FIG. 6 is a perspective cross-section view an electroporation cuvette 45 having a deepened well 54 (the cuvette 45 being shown with the cover 36 removed). As with the cuvette of FIG. 1, there is a body 46 having a quadratic form, and an upper portion 48 having a circular form. The body 46 defines a cavity 50 at least partially defined by an angled wall 52. A well 54 is provided at the very bottom of cavity 50. Well 54 can have a shape different from the well 28 of FIG. 1, where the well contains vertical walls 56 and angled walls 57. The vertical walls 56 can be extended according to the volume of sample intended to be accommodated by the well 54. For example, the vertical walls 56 can be extended to accommodate a volume of 100 μL. When the thickness of the well is about 0.1 cm, the vertical wall can be about 8.7 mm in order to accommodate a 100 μL sample. The vertical walls 56 can extend further, or can be shorter, according to the volume of sample intended to be accommodated. Exemplary sample volumes with a 0.1 cm well thickness include about 150 μL, about 125 μL, about 100 μL, about 80 μL, about 60 μL, about 50 μL, about 45 μL, or about 40 μL.

Thus, while a shallow well represents an exemplary configuration for accommodating frozen cells, it is contemplated herein that any well of a cuvette, including deepened wells, can be used for accommodating frozen cells in the methods, apparatuses and combinations provided herein.

FIG. 7 provides another exemplary configuration of a cuvette. FIG. 7 depicts a circular top portion 114 of cuvette 110 and a body 112, which contains cavity 122. Cavity 122 has one or more side walls 124 which are connected at their bottom portion to angled walls 126, which are downwardly tapered and connect at their bottom portion to a well 128. Similar to the previously described embodiments, the well 128 is at least partially defined by opposing walls that are downwardly angled toward one another such that the well 128 tapers to a vertex. Downwardly angled walls of the V-shaped well 128 are oriented substantially perpendicular to the intermediate tapered region of the cavity 122 (formed by angled walls 126). The cuvette 110 may include an inset 132 and a projection 130 for stabilizing and/or positioning the cuvette. The cuvette 110 may also include cover 118 which can be used to prevent substances outside of the cuvette from entering the cuvette and substances inside the cuvette from separating from the cuvette.

In this embodiment, the cuvette 110 also contains electrodes 129 which are substantially “J” shaped. In such embodiments, the electrode 129 may traverse along the outside walls of cuvette 110 and may extend inwardly into the cuvette below the well, such as along or near the cuvette bottom. The electrode 129 may ascend within the cuvette along the opposing parallel walls that at least partially define the well 128. As such, the electrodes 129 may be configured such that portions of two opposed surfaces 131, one from each electrode 129, at least partially define opposite sides of well 128 along the thickness dimension of the well 128.

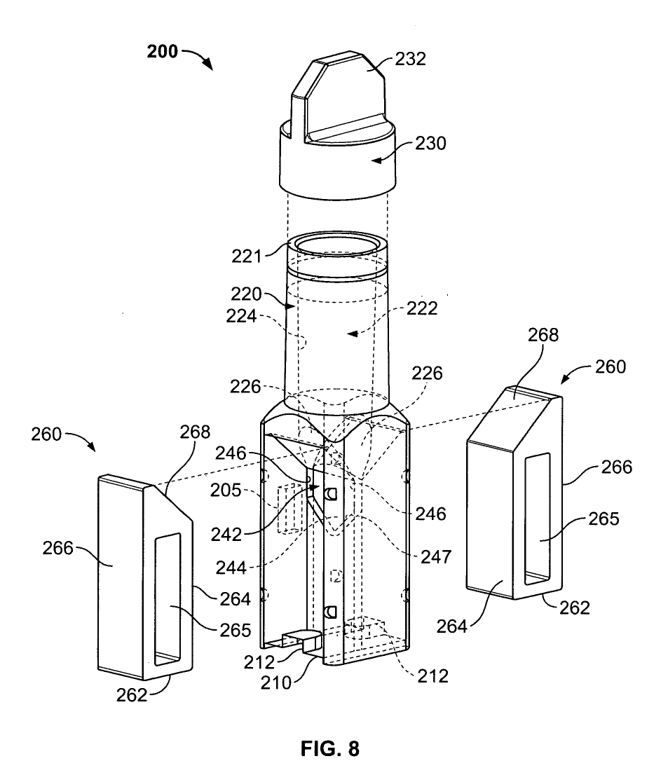

Referring now to FIGS. 8-10, a cuvette 200 may include a top portion 220 having a substantially circular cross-sectional shape (e.g., having a substantially cylindrical or conical shape). The top portion 220 may join with a body 240 having a substantially quadratic shape, such as a square cross-sectional shape with substantially rounded corners. In some circumstances, the rounded corners of the cuvette body 240 may provide for a proper fit of the cuvette 200 into an electroporator device. Similar to the previously described embodiments, the cuvette 200 may include at least one projection 205 and an inset 210 on the bottom surface of body 240, which are capable of providing proper seating and/or orientation of cuvette 200 when it is placed in an electroporator device. One or more channels 212 may be formed proximal to the bottom of the cuvette body 240 such that bottom surfaces 262 of the electrodes 260 may be contacted from the bottom side of the cuvette body 240. In such circumstances, an electroporator device may form electrical contacts with the bottom surfaces 262 of the electrodes 260 in addition to, or as an alternative to, electrical contacts with the exterior side surfaces 266 of the electrodes 260. A cover 230 may be configured to releasably engage the top portion 220 of the cuvette 200. The cover 230 may include an extension tab 232 that may be grasped by a user to manipulate the cover 230 or to remove the cover 230 from the top portion 220 (refer also to FIG. 15). In some embodiments, the cover 230 may engaged the top portion 220 such that the entire cuvette 200 may be lifted or adjusted by grasping and maneuvering the extension tab 232 of the cover 230.

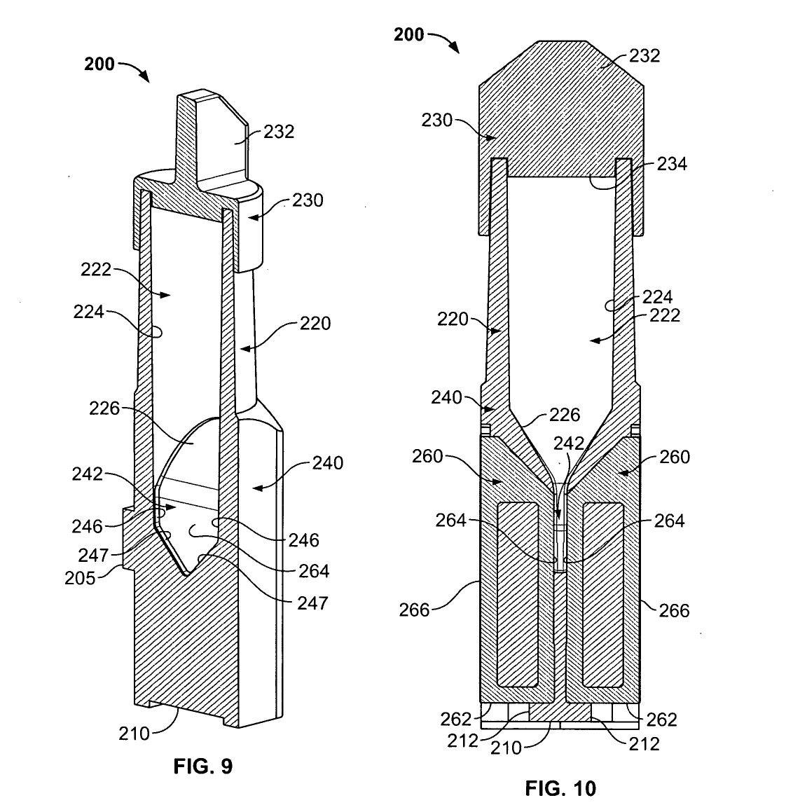

As shown in FIGS. 8-9, the cuvette 200 may include a cavity 222 that extends downwardly through the top portion 220. The cavity 222 may be at least partially defined by a side wall 224 have a circular cross-sectional shape, and the cavity 222 may be at least partially defined by two intermediate walls 226 that are generally downwardly angled toward one another. Each of the intermediate walls 226 may have a curved surface that generally extends at a downward angle toward a well 24, which is disposed at the bottommost portion of the cavity 222. The well 242 may be adapted to contain electrocompetent cells in a space between the two electrodes 260. The well 242 may be at least partially defined by two opposing parallel walls 244, each of wall may comprise a portion of an interior facing surface 264 of an electrode 260. Similar to the well 54 of FIG. 6, the well 242 may be at least partially defined by substantially vertical walls 246 and angled walls 247. The vertical walls 246 may be extended according to the desired volume of sample to be accommodated by the well 242. The angled walls 247 of the well 242 may oppose one another such that the walls 247 are downwardly angled toward one another. Accordingly, in this embodiment, the walls that substantially define the cavity 222 (including the well 242) provide a smooth transition from the upper portion of the cavity 222 to the well 242 and are generally non-parallel to the horizontal upper rim 221 of the top portion 220. In the embodiment depicted in FIGS. 8-10, the direction of general slope of intermediate walls 226 oriented substantially perpendicular to the direction of slope of the angled walls 247 in the well 242. As such, the intermediate walls 226 may funnel a liquid sample into the well 242 while the angled walls 247 serve to direct at least a portion of the liquid sample to a single lowest region of the well 242.

In the embodiment depicted in FIGS. 8-10, the angled walls 247 join at a rounded vertex so that the bottommost portion of the well 242 has a V-shape. Each angled wall 247 may have a flat planar surface. In such circumstances, the angled walls 247 of the well 242 may slope downwardly at angles varying from 30-60 degrees, such as 40-50 degrees, including 45 degrees, relative to the substantially vertical walls 247 of the well 242. In other embodiments, each angled wall 247 may have a curved surface that generally extends at a downward angle toward the opposing angled wall 247. For example, the angled walls 247 of the well 242 may have an arcuate shape such that the bottommost portion of the well 242 is substantially shaped as a downwardly extending arc or semicircle.

As previously described, a well having a downwardly tapered shape, such as the V-shaped bottom portion of the well 242 may provide several advantages. For example, the tapered well shape can make it easier for a user to access materials in the well 242. In particular, if the bottom of the well 242 was flat or substantially horizontal, a user would have to reach into all of the corners to obtain the material contained in the well 242, but the tapered well shape may operate as a funnel to direct the material to a single lowest region where it can then be removed from the well 242. The tapered well shape also guides the tip of any removal instrument toward the bottom portion of the well 242, where the material is likely positioned. The tapered well shape may also help prevent air from becoming lodged in the cuvette, which may induce electrical arcing across the well 242 during electroporation operations.

Still referring to FIGS. 8-10, in some embodiments, the length of the of the cuvette 200, from the bottom of body 240 to the upper rim 221 of top portion 220 can be about 45.1 mm. In such embodiments, the top portion may have a substantially circular cross-sectional shape with a diameter of about 10.7 mm near the upper rim 221. The body 240 can be substantially quadratic in cross-section, with a dimension of about 12.2 mm by 12.1 mm. The upper portion of the cavity 222 may be substantially circular in cross-section, with a cavity diameter of about 8.5 mm near the upper rim 221. In some cases, the cavity diameter may taper to a larger size as the cavity extends downward toward the intermediate angle walls 226. The side wall 224 of cavity 222 can be about 20.7 mm long, and the intermediate angled walls 226 can be 4.9 mm long. In some embodiments, the portion of the cavity 222 above the well 242 may be adapted to accommodate a liquid sample of at least about 500 μL, at least about 700 μL, at least about 900 μL, at least about 1,100 μL, or at least about 1,300 μL. For example, in the embodiment depicted in FIGS. 8-10, the portion of the cavity 222 above the well 242 may be adapted to accommodate a liquid sample of about 1,100 μL The well 242 may be described by any of a number of dimensions, including thickness of the well 242, width of the well and depth of the well, or volume of the well, or the cross sectional area to depth ratio of the well, or the ratio of the longer of the width or thickness relative to the depth of the well. As previously described, the thickness of the well 242 may represent the dimension of the well that separates the two electrodes (e.g., the gap space between the electrodes). The thickness can be any of a range of sizes according to the volume of sample to be accommodated in the well and according to the intended separation of the electrodes. In one embodiment, the thickness of the well is selected according to the intended separation of the electrodes 260. For example, the thickness can range from about 0.05 cm to about 0.5 cm, from about 0.08 cm to about 0.4 cm, from about 0.1 cm to about 0.3 cm, from about 0.1 cm to about 0.3 cm. Exemplary thicknesses are about 0.1 cm or about 0.2 cm.

The width of the well 242 can be any size that can be formed within an electroporation vessel such as an electroporation cuvette and also serves to accommodate the intended sample volume and, optionally, that has the intended size relative to the well depth. As previously described, the bottommost portion of the well 242 may be downwardly tapered, such that the width is largest between the substantially vertical walls 246 and smallest at the bottom of the well 242. In some embodiments, the width 242 at the top of the well 242 (e.g., between the substantially vertical walls 246) can range from about 10 mm to about 6 mm, and can taper to a vertex at the bottom of the well. For example, the well width between the substantially vertical walls 246 may be about 7.3 mm or about 7.5 mm. In such embodiments, the width to depth ratio can be 1:1.5, 1:1.0, 1:0.7, 1:0.5, 1:0.4, 1:0.3, or 1:0.2. In another example, the width can be of a size such that the horizontal cross sectional area at the top of the well 242 (width×thickness) is at least about 3.0 mm 2, at least about 5.0 mm², at least about 8.0 mm², at least about 10.0 mm², at least about 12.0 mm², at least about 15.0 mm², at least about 20.0 mm², at least about 30.0 mm², or at least about 40.0 mm². Exemplary horizontal cross-sectional areas at the top of some wells may be about 7.3 mm², about 7.6 mm², or about 15.0 mm².

The depth of the well 242 can be any size that can be formed within an electroporation vessel such as an electroporation cuvette and also serves to accommodate the intended sample volume and, optionally, that has the intended size relative to the well width. The thickness of the well 242 (e.g., the gap space between the electrodes 260) may be sized to receive the tip portion of a pipette, which may be used to deposit or withdraw material from the well 242. As such, a user may operate a pipette to extend into the well 242 to extract a liquid sample from the bottom portion of the well 242. If the thickness of the well 242 is increased for a particular embodiment, the depth can be decreased while still accommodating the intended volume. The vertical walls 246 may be extended according to the desired volume of sample to be accommodated by the well 242. Exemplary depths of some wells may be at least about 2.5 mm, at least about 5.1 mm, at least about 10.0 mm, at least about 16.0 mm, or at least about 20.0 mm.

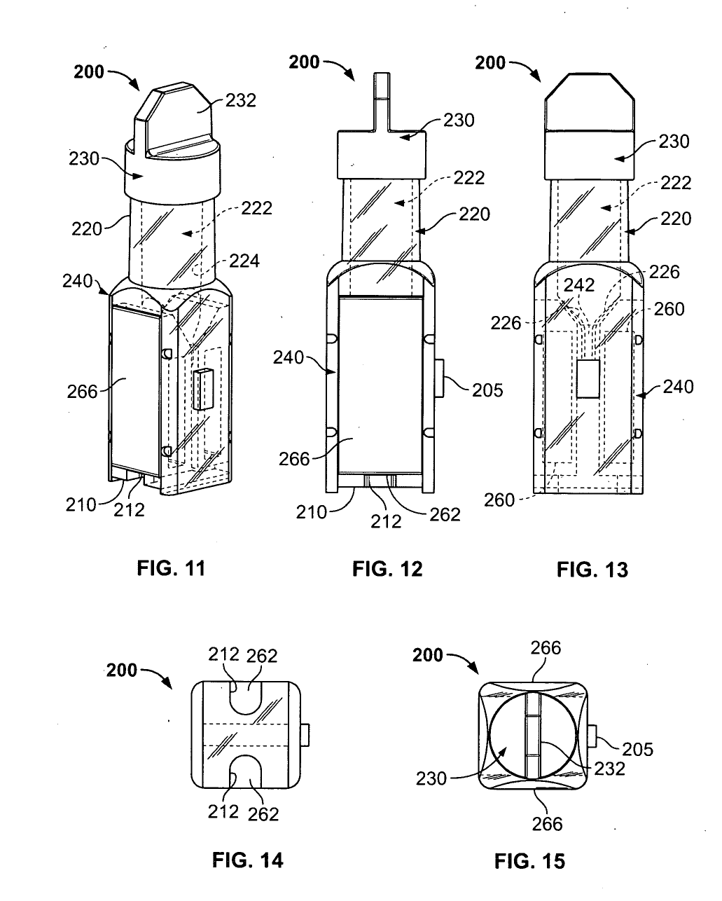

Referring to FIGS. 8-10 and to FIGS. 11-13, the cuvette 200 may include electrodes 260 that are configured to pass electrical energy into the well 242. Each electrode 260 may include an interior surface 264, a portion of which at least partially defines the well 242 and is adapted to contact material, such as a cell suspension, in the well 242. The opposing interior surfaces 264 of the electrodes 260 may be spaced apart by a predetermined gap, which can establish the thickness of the well 242. The electrodes may include an exterior surface 266 that is substantially aligned with an outer surface of the cuvette body 240 (as shown, for example, in FIG. 10 and FIG. 13). The exterior surfaces 266 of the electrodes 260 may serve as electrical contacts that abut associated contacts on the electroporator device. According, the electroporator device may apply a voltage may be applied across the well 242 through the electrical contacts that abut the exterior surfaces 266 of the electrodes 260. The exterior surfaces 266 may have different configurations depending upon the associated electrical contacts on the electroporator device. In one example, the exterior surface 266 of each electrode 260 may provide a contact area that is about 20.8 mm in height and about 8.6 mm in width.

Referring to FIGS. 10, 12, and 14, as previously described, one or more channels 212 may be formed proximal to the bottom of the cuvette 200 such that bottom surfaces 262 of the electrodes 260 may be electrically contacted from the bottom side of the cuvette body 240. In such circumstances, an electroporator device may form electrical contacts with the bottom surfaces 262 of the electrodes 260 in addition to, or as an alternative to, electrical contacts with the exterior surfaces 266 of the electrodes 260. Accessing the electrodes 260 from the bottom side may provide for a safe design of the electroporator device by reducing the likelihood of inadvertently touching the electrical contacts in the electroporator device. Furthermore, the channels 212 may provide another mechanism for properly orienting and seating the cuvette 200 in the electroporator device.

Referring again to FIGS. 8-10, each electrode 260 may be configured to have a lateral channel 265 formed therethrough. In addition, the electrode 260 may have a downwardly angled surface 268 that extends between the exterior surface 266 and the interior surface 264. In such embodiments, the exterior surface 266 may provide a relatively large contact area while the contact areas of the well 242 are disposed at a lower position. As such, a greater volume of material may accommodated in the upper portion of the cavity 222 above the well 242. The electrode 260 may be formed by extruding a material through a die that defines the lateral channel 265 and the downwardly angled surface 268. The extruded rod may be cut to the selected width to form the electrodes as shown in FIG. 8. The electrodes 260 can be manufactured from metallic or otherwise electrically conductive materials, such as aluminum, stainless steel, copper, gold, or silver. For example, the electrodes 29 may comprise a 6063-T6 aluminum alloy. In some cases, the electrodes may be finished by plating with gold, silver, copper, or zinc.

As shown in FIG. 10, the cuvette body 240 may be formed to retain the electrodes 260 in a desired position. For example, the electrodes 260 may be integrally molded into the cuvette during an injection molding process to form the cuvette body 240. In such cases, the cuvette body 240 may be shaped as shown in FIG. 9 while a portion of the cuvette body material passes into the lateral channels 265 of the electrodes 260. When the cuvette body material is hardened, the electrodes are secured in the desired position because a portion of the cuvette body material is filled into the lateral channels 265 and joined with the cuvette body walls (refer to FIG. 10). The cuvette body may be formed from a polymer material, such as SAN (styrene acrylonitrile), Polycarbonate, Polystyrene, Acrylic, PMMA (polymethyl methacrylate), PET (polyethylene terephthalate), PETG (polyethylene terephtalate glycol), or polypropylene. In some embodiments, the cuvette body may be formed of a substantially transparent material so that the tools or liquid sample inside the cavity 222 may be readily viewed by a user. As described in more detail below, the cuvettes 200 may contain frozen cells. In such circumstances, the cuvette body may be capable of withstanding temperatures of up to about −40° C., up to about −60° C., or up to about −80° C. without substantial cracking for storage periods of at least about 30 days, at least about 60 days, at least about 90 days, or at least about 120 days. Exemplary embodiments of the cuvette body may be capable of withstanding temperatures of −90° C. without substantial cracking for a period about 30 minutes and may be capable of withstanding temperatures of −80° C. for at least 30 days without substantial cracking.

The electrodes 260 can be spaced apart by any of a variety of distances suitable for the cells to be electroporated. For example, the gap space between the electrodes 260 may be sufficiently large so as to receive the tip portion of a pipette, which may be used to deposit or withdraw material from the well 242. As such, a user may operate a pipette to extend into the well 242 to extract a liquid sample from the bottom portion of the well 242. Suitable spacings of electrodes for different cells are known in the art and are readily available from any of a variety of sources. For example, the gap space (e.g., the well thickness) can range from about 0.08 cm to about 0.5 cm, from about 0.1 cm to about 0.4 cm, from about 0.2 cm to about 0.3 cm, or from about 0.22 cm to about 0.28 cm. Exemplary gap spacing between electrodes 260 may be about 0.1 cm or about 0.2 cm, depending upon the desired sample volume to be accommodated and the well width and depth.

Referring to FIGS. 12-13 and 15, the cover 230 may have a substantially circular cross-sectional shape to mate with the top portion 220 of the cuvette 200. Because the outer diameter of the top portion 220 may be smaller than the width of the body 240, the cover 230 may be configured to have an outer diameter that is smaller than, or substantially similar to the outer width of the body 240. As such, the cuvette 200 may be positioned substantially adjacent to a neighboring cuvette 200 without interference from the outer rim of the cover 230. By providing an opportunity for closer spacing of the neighboring cuvettes, handling of groups of cuvettes may be more efficient.

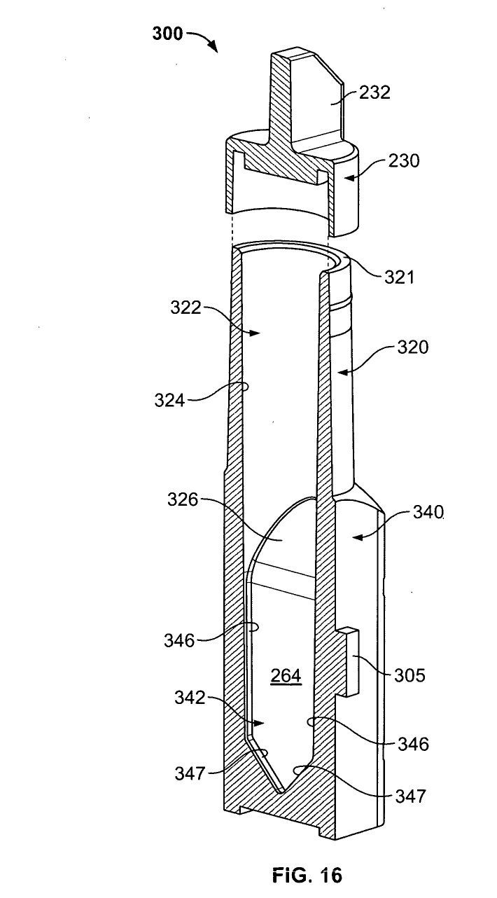

As previously described, the well of the cuvette may be at least partially defined by substantially vertical walls and angled walls, and the vertical walls may be extended according to the desired volume of sample to be accommodated by the well. Referring now to FIG. 16, some embodiments of a cuvette 300 may include a deepened well 342 that extends to a depth proximal to the bottom of the cuvette. The deepened well 342 may be adapted to contain electrocompetent cells in a space between the two electrodes, such as electrodes 260 (FIG. 8). The well 342 may be at least partially defined by two opposing parallel walls, each of Wall may comprise a portion of an interior facing surface 264 of an electrode 260. The well 342 may be at least partially defined by substantially vertical walls 346 and angled walls 347. The angled walls 347 of the well 342 may oppose one another such that the walls 347 are downwardly angled toward one another. Comparing the vertical walls 346 (FIG. 16) to the vertical walls 246 (FIG. 9), the vertical walls 346 are extended to provide a deeper well 342. The cuvette 300 may include a deepened well 342 so as to accommodate a greater volume of liquid, to expose a greater portion of the interior electrode surfaces 264, or both.

Similar to the embodiments previously described in connection with FIGS. 8-10, the cuvette 300 may include a cover 230, a top portion 320, a cavity 322, intermediate angled walls 326, a body 340, and a well 342 disposed between opposing electrodes 260. The top portion 320 may have a substantially circular cross-sectional shape. The top portion 320 may join with a body 340 having a substantially quadratic shape, such as a square cross-sectional shape with substantially rounded corners. The rounded corners of the cuvette body 340 may provide for a proper fit of the cuvette 300 into an electroporator device. Also, the cuvette 300 may include at least one projection 305 and an inset on the bottom surface of body 340, which are capable of providing proper seating and/or orientation of cuvette 300 when it is placed in an electroporator device. The cuvette 300 may include one or more channels (refer, for example, to channels 212 in FIG. 8) formed proximal to the bottom surface of body 340 such that bottom surfaces 262 of the electrodes 260 (FIG. 8) may be contacted from the bottom side of the cuvette body 340. In such circumstances, an electroporator device may form electrical contacts with the bottom surfaces 262 of the electrodes 260 in addition to, or as an alternative to, electrical contacts with the exterior surfaces 266 of the electrodes 260 (described in more detail below). The cover 230 may be configured to have an outer diameter that is smaller than, or substantially similar to, the outer width of the body 340. As such, the cuvette 300 may be positioned substantially adjacent to a neighboring cuvette 300 without interference from the outer rim of the cover 230.

The thickness of the well 342 may represent the dimension of the well that separates the two electrodes (e.g., the gap space between the electrodes). The thickness can be any of a range of sizes according to the volume of sample to be accommodated in the well and according to the intended separation of the electrodes. In one embodiment, the thickness of the well is selected according to the intended separation of the electrodes 260. For example, the thickness can range from about 0.05 cm to about 0.5 cm, from about 0.08 cm to about 0.4 cm, from about 0.1 cm to about 0.3 cm, from about 0.1 cm to about 0.3 cm. Exemplary thicknesses are about 0.1 cm or about 0.2 cm.

The width of the well 342 can be any size that can be formed within an electroporation vessel such as an electroporation cuvette and also serves to accommodate the intended sample volume and, optionally, that has the intended size relative to the well depth. As previously described, the bottommost portion of the well 342 may be downwardly tapered, such that the width is largest between the substantially vertical walls 346 and smallest at the bottom of the well 342. In some embodiments, the width 342 at the top of the well 342 (e.g., between the substantially vertical walls 346) can range from about 10 mm to about 6 mm, and can taper to a vertex at the bottom of the well. In exemplary embodiments, the well width between the substantially vertical walls 246 may be about 7.3 mm or about 7.5 mm. In such embodiments, the width to depth ratio can be 1:1.5, 1:1.0, 1:0.7, 1:0.5, 1:0.4, 1:0.3, or 1:0.2. In another example, the width can be of a size such that the horizontal cross sectional area at the top of the well 342 (width×thickness) is at least about 3.0 mm², at least about 5.0 mm², at least about 8.0 mm², at least about 10.0 mm², at least about 12.0 mm², at least about 15.0 mm², at least about 20.0 mm², at least about 30.0 mm², or at least about 40.0 mm². Exemplary horizontal cross-sectional areas at the top of some wells may be about 7.3 mm², about 7.6 mm², or about 15.0 mm².

The depth of the well 342 can be any size that can be formed within an electroporation vessel such as an electroporation cuvette and also serves to accommodate the intended sample volume and, optionally, that has the intended size relative to the well width. The thickness of the well 342 (e.g., the gap space between the electrodes) may be sized to receive the tip portion of a pipette, which may be used to deposit or withdraw material from the well 342. As such, a user may operate a pipette to extend into the well 342 to extract a liquid sample from the bottom portion of the well 342. The vertical walls 346 may be extended according to the desired volume of sample to be accommodated by the well 342. In the embodiment shown in FIG. 16, the depth of the well 342 may be about 15.9 mm. It should be understood that the depth of the well 342 may be adjusted to accommodate different volumes of liquid samples in the well 342.

In some embodiments, the dimensions of the well 342 may be determined according to the volume of liquid the well 342 an accommodate. Exemplary wells can accommodate a 35 μL liquid sample, a 110 μL liquid sample, or a 220 μL liquid sample. Well volume can vary as a function of the thickness of the well 342. In some examples, a well 242 with thickness of 0.1 cm can accommodate a volume of liquid up to about 35 μL or up to about 110 μL, depending upon the selected well width and depth. In one embodiment, well 342 can be configured such that when accommodating sample, such as, for example, a 35 μL sample, the horizontal cross-sectional area to volume ratio of the sample is about 217 m⁻¹. In another embodiment, well 342 can be configured such that when accommodating sample, such as, for example, a 110 μL sample, the horizontal cross-sectional area to volume ratio of the sample is about 66 m⁻¹. In a further example, a well 342 with thickness of 0.2 cm can accommodate a volume of liquid up to about 220 μL, depending upon the selected well width and depth. In one embodiment, well 342 can be configured such that when accommodating sample, such as, for example, a 220 μL sample, the horizontal cross-sectional area to volume ratio of the sample is about 68 m⁻¹. In other embodiments that accommodate a 35 μL sample, a 110 μL sample, or a 220 μL sample, the horizontal cross-sectional area to volume ratio of the sample may be about or greater 50 m⁻¹ or greater, 100 m⁻¹ or greater, 150 m⁻¹ or greater, 200 m⁻¹ or greater, 250 m⁻¹ or greater, 300 m⁻¹ or greater, 400 m⁻¹ or greater, or 500 m⁻¹ or greater.

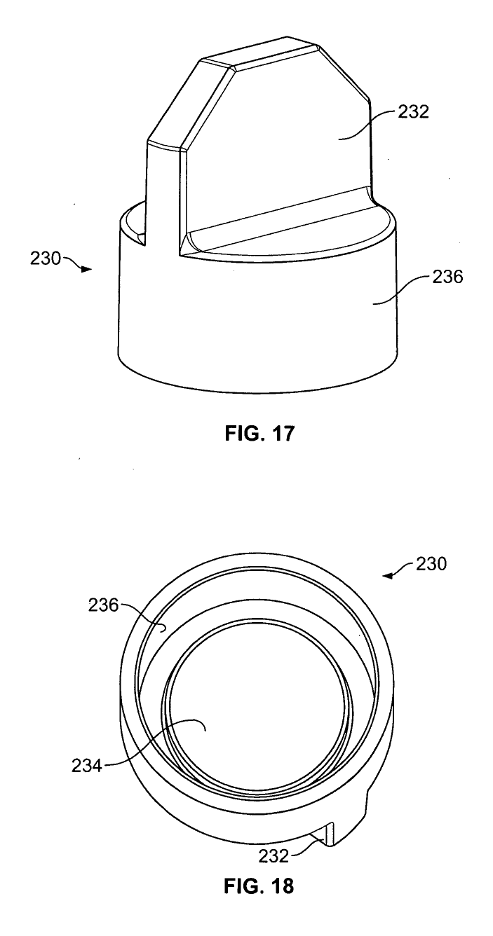

Referring now to FIGS. 17-18, the cover 230 may be releasably attachable to a cuvette (e.g., cuvette 110, cuvette 200, or cuvette 300) so as to effectively seal the contents of the cuvette. As previously described, cover 230 is provided with an extension tab 232 that extends in a substantially upward direction. The extension tab 232 is configured to be readily grasped by a user's thumb and finger for removal of the cover 230 from a cuvette. The extension tab 232 may extend in a generally upward direction so that neighboring cuvettes 200 to permit side-by-side placement of neighboring cuvettes. For example, neighboring cuvettes may be placed adjacent to one another without interference from laterally extending members on the cover. The cover 230 may include an interior circumferential lip 236 to engage a shoulder of the cuvette, which may secure cover 230 more tightly on the cuvette. The interior circumferential lip 236 can be continuous or can have multiple extensions separated by gaps (not shown in FIGS. 17-18). Such gaps between portions of the interior circumferential lip 236 may permit air to escape as the cover 230 is being placed on and/or being removed from a cuvette. The cover 230 may include a descending portion 234 that is configured to correspond to the inside shape and size of the cuvette cavity. In this embodiment, the descending portion 234 has a substantially cylindrical shape that correspond to the substantially circular cross-sectional shape of the top portion of the cuvette's cavity. The descending portion 234 can be molded as a single piece with the rest of cover 230, or could be attached to, or otherwise molded into, the cover 230.

In some alternative embodiments, the exterior of a top portion of the cuvette can also be provided with threads, whether formed as threads that extend from top portions, or that are formed cut into the top portion. In such embodiments, the cover may be provided with corresponding threads to allow for a screw-on cap. In another alternative embodiment, the cover may be a plug that fits inside the top portion of the cuvette's cavity. The plug can be provided with a shoulder portion that is larger than the cavity of the cuvette to prevent the plug from descending to far into the cuvette's cavity.

B. Preparing Cuvette with Frozen Cells and Use Thereof

As provided in the methods, apparatuses and combinations disclosed herein, a frozen cell suspension can be packaged in an electroporation vessel, such as the electroporation vessel described herein, as exemplified by cuvettes 10, 110, 200, and 300. For example, the suspension can be added to the electroporation vessel and then frozen. In another example, the suspension can be frozen and then added to the electroporation vessel while in frozen form. The cells can then be stored in frozen form and, optionally, transported to a separate site for use. At the time of use, the cover and/or foil layer 16, if present, can be removed from the vessel, and exogenous material can be added to the cavity of the vessel after the cell suspension has been allowed to thaw adequately. In another example, at the time of use, the cover and/or foil layer 16, if present, can be removed from the vessel, and exogenous material can be added to the cavity of the vessel before the cell suspension has thawed. The added exogenous material can be mixed by any of a variety of methods known in the art such as drawing solution in and out of a pipette or tapping the cuvette with a finger.

1. Preparation of Electrocompetent Cells

Cells used in the methods, apparatuses and combinations provided herein can be any of a variety of cells known in the art to be transformed using electroporation methods. Any of a variety of conditions can be used for preparing electrocompetent cells, as known in the art.

a. Cell Types

Cells in accord with the methods provided herein are isolated (i.e., separated at least partially from other bacteria and materials with which they are associated in nature) and are rendered electrocompetent. Any isolated cells, prokaryotic or eukaryotic, can be used. Methods for rendering prokaryotic and eukaryotic cells electrocompetent are well known and can be practiced as a matter of routine by those skilled in the art in the art. Any method will suffice, and none is particularly preferred.

Bacterial cells suitable for the methods, apparatuses and compositions provided herein include gram negative and gram positive bacteria of any genus. Exemplary bacteria include, but are not limited to, Escherichia sp. such as E. coli; Klebsiella sp.; Streptomyces sp.; Streptocococcus sp.; Shigella sp.; Staphylococcus sp.; Erwinia sp.; Bacillus sp. such as B. cereus, B. subtilis and B. megaterium; Serratia sp.; Pseudomonas sp. such as P. aeruginosa and P. syringae; and Salmonella sp. such as S. typhi and S. typhimurium.

Any of a variety of bacterial strains and serotypes known in the art can be used in the methods, apparatuses and compositions provided herein, including E. coli strains K, B, C, and W. An exemplary bacterial host is E. coli strain K-12.

Any of a variety of yeast or fungal cells also can be rendered electrocompetent and used in the methods, apparatuses and compositions provided herein. Exemplary yeast and fungi include, but are not limited to, Aspergillus sp., Candida sp., Colletotrichum sp., Cryptococcus sp., Dictyostelium sp., Pichia sp. including Pichia pastoris, Saccharomyces sp. including Saccharomyces cerevisiae, and Schizosaccharomyces sp. Any of a variety of plant cells also can be rendered electrocompetent and used in the methods, apparatuses and compositions provided herein. Exemplary plant cells include, but are not limited to, algae, maize, tobacco, wheat, rice, and arabidopsis. Any of a variety of animal and mammalian cells also can be rendered electrocompetent and used in the methods, apparatuses and compositions provided herein. Exemplary animal cells include, but are not limited to, primate including human and monkey, rodent including mouse, rat and hamster, sheep, cow, horse, goat, bird including chicken.

b. Cell Preparation

Cells included in the methods, apparatuses and combinations provided herein are made electrocompetent prior to introducing the cells into the electroporation vessel such as the electroporation cuvette exemplified herein. A wide variety of methods for preparing electrocompetent cells are known in the art, and can be used to prepare the cells provided herein.

For exemplary purposes, electrocompetent cells can be prepared according to the method provided in U.S. Pat. No. 5,891,692 to Bloom et al. and U.S. Pat. No. 4,981,797 to Jessee et al. In this example, a single colony isolate such as an isolate of E. coli strain DH10B can be made electrocompetent. Essentially the process is as follows: The single colony isolates can be inoculated into 2 ml of SOB minus Mg²⁺ media (2.0% tryptone, 0.5% yeast extract, 10 mM NaCl, 2.5 mM KCl, 0.005% polypropylene glycol (PPG)) and shaken overnight at 225 rpm at a temperature of 37° C. To a baffled flask containing 1 liter of SOB minus Mg²⁺ media can be added 4 ml of streptomycin at 100 μg/ml and an 0.8 ml aliquot of overnight culture. The resulting cultures can be grown by shaking them at 225 rpm at a temperature of 37° C. until the O.D.₅₅₀ of the cultures is approximately 0.3. The culture can be harvested by centrifugation of at 5,000 rpm and 2° C., for a sufficient time to pellet the bacterial cells. The bacterial cell pellets can be then resuspended in 4 ml of ice cold CCMB80 buffer (10 mM potassium acetate pH 7.0, 80 mM CaCl₂, 20 mM MnCl₂, 10 mM MgCl₂, 12% glycerol adjusted to pH 6.4 with 0.1N HCl, as described in Hanahan, et al., Methods in Enzymology, 204:63-113 (1991), herein incorporated by reference. The resuspended bacterial cells can be then kept on ice for 20 minutes. The resuspended bacterial cells can be then divided into aliquots and frozen or placed into an electroporation vessel and frozen, according to the methods provided herein.

A variety of additional methods for preparing competent cells are readily available to those skilled in the art. Exemplary sources of additional protocols for preparation of electrocompetent cells can be found, for example, in Protocols in Molecular Biology by Ausubel et al., and available at online sites such as http://www.protocol-online.org/prot/Molecular Biology/DNA/Transformation/Electroporation/and http://www.bio-rad.com/LifeScience/pdf/New_Gene Pulser.pdf.

c. Freezing the Cells

Electrocompetent cells prepared according to the exemplary method provided herein, or by any other method known in the art can be frozen. In accordance with the methods, apparatuses and combinations provided herein, freezing cells can permit cells to be stored for long time periods and also can facilitate transit of the cells into an electroporation vessel without disrupting accurate placement of the cells in the intended electroporation vessel compartment. For example, electrocompetent cells can be placed in liquid form into an electroporation vessel, such as the electroporation cuvette exemplified herein, and then frozen. The liquid cell suspension may be placed in contact with at least one electrode, and often two electrodes of the electroporation vessel.

The liquid cell suspension can also be placed in the electroporation vessel and, optionally subjected to additional manipulation steps to achieve an intended form. Additional manipulation steps include steps where the liquid cell suspension can be, for example, let to stand or agitated in order to remove any gaseous bubbles that may be present in the liquid cell suspension, prior to freezing. It can be desirable to remove air bubbles from a suspension of electrocompetent cells prior to performing any electroporation steps in order to prevent arcing or other unintended results that can decrease electroporation efficiency. By performing one or more steps of removing gaseous bubbles in the liquid cell suspension prior to freezing, the need for gaseous bubble removal is reduced at the point of use after thawing and prior to electroporation. Thus gaseous bubble removal prior to freezing can further act to decrease the time required for electroporation at the point of use, and can also provide the benefit of insuring a higher quality suspension (e.g., without gaseous bubbles) than may otherwise arise at the point of use, which may result in higher electroporation efficiency.

The volume of liquid cell suspension added to the electroporation vessel can be any of a wide range of volumes that can be used in electroporation vessels, including the volumes of electroporation vessels provided herein. In one embodiment, the volume can be 400 μL or less, about 360 μL or less, about 320 μL or less, about 280 μL or less, about 240 μL or less, about 200 μL or less, about 180 μL or less, about 160 μL or less, about 140 μL or less, about 120 μL or less, about 100 μL or less, about 80 μL or less, about 70 μL or less, about 60 μL or less, about 50 μL or less, about 45 μL or less, about 40 μL or less, about 35 μL or less, about 30 μL or less, or about 25 μL or less. In one example, the volume is about 35 μL. In one embodiment, the same volume of liquid cell suspension is added to a plurality of electroporation vessels, where the same volume can refer to aliquots that vary in volume by about 10% or less, about 5% or less, about 3% or less, about 2% or less, or about 1% or less.

The liquid cell suspension can be frozen in the electroporation vessel. In one embodiment, the liquid cell suspension is frozen at the location in the vessel in which the electroporation procedure is to be performed. In another embodiment, the liquid cell suspension is frozen at a location in the vessel in which the liquid cell suspension is in contact with at least one electrode or with two electrodes. In another embodiment, the liquid cell suspension is frozen in a well of the electroporation vessel such as the well described herein, where the well can be, for example, a shallow well as described herein. For example, the cells can be frozen in V-shaped well 28 of FIG. 1 or V-shaped well 128 of FIG. 7.

After any steps performed on the liquid cell suspension in the electroporation vessel, the liquid cell suspension can be frozen. Any of a variety of procedures for freezing liquids, particularly procedures for freezing electrocompetent cells, can be used. In one example, the liquid cell suspension can be frozen by placing the cuvette containing the liquid cell suspension into a dry ice/ethanol bath for at least 5 minutes. The liquid cell suspension can be frozen by placing the liquid cell suspension into a pre-cooled electroporation vessel, or by placing the cells in an electroporation vessel at a temperature above freezing, such as room temperature, 4° C., or on ice (at about 0° C.). Liquid cell suspension placed into an electroporation vessel at a temperature above freezing can then be frozen by placing the electroporation vessel into freezing conditions. In an alternate embodiment, the liquid cell suspension can be frozen outside of the electroporation vessel, and then can be added to the electroporation vessel in frozen form. In one such example, the cells can be frozen in a mold shaped similarly to the shape of a portion of the electroporation vessel in such a way that the frozen cells can be added to the vessel and accommodated by the similarly shaped portion of the electroporation vessel (e.g., the electroporation well).

Freezing conditions can take any of a variety of forms that cool by contact with a solid, liquid or gas at or below freezing temperatures. For example, freezing conditions can be an aluminum block at or below freezing temperatures, a liquid at or below freezing temperatures such as an ethanol/dry ice bath, or a freezer at or below freezing temperatures, including devices designed for rapid and/or controlled freezing, such as cryofreezers. Freezing temperature can range from below about 0° C. to about −180° C., about −20° C. to about −120° C., such as −90° C. The cell suspension is maintained under freezing conditions for at least as long as required for the cell suspension to freeze, and can also be long enough for the cell suspension to reach the same temperature as that of the freezing conditions. The time length for maintaining the cell suspension under freezing conditions can vary according to the amount freezing conditions, and can readily be determined by one skilled in the art. The cell suspension may be maintained under freezing conditions for at least 30 second, at least 1 minute, at least 2 minutes, at least 5 minutes, at least 10 minutes, at least 20 minutes, at least 30 minutes, or at least 1 hour. In some embodiments, the cell suspension can be stored for longer time periods under the freezing conditions.

Exemplary conditions for freezing electrocompetent cells at −90° C. temperatures are as follows. Into an electroporation cuvette is placed 35 μl of electrocompetent cells, and the cuvette is capped. The cuvette is then placed into a cryofreezer at −90° C. for 10 minutes.

d. Cell Storage

Electroporation vessels containing frozen cell suspension can be stored for any amount of time and under any conditions in which the cells do not appreciably lose their transformation efficiency and/or viability. For example, cells can be stored for a time and under conditions in which cells maintain about 40% to 100%, about 60% to 100%, about 70% to 100%, or about 80% to 100% of their original transformation efficiency and/or viability. For example, cells can be stored for any amount of time that permits a yield of about 1×10⁶ transformants, or more. Suitable storage temperatures for the cells vary from below about 0° C. to about −180° C. The storage temperature may range from about −20° C. to about −120° C., or about −20° C. to about −90° C., e.g., −80° C. The storage period or time can range from about 0 days to about 180 days (e.g., 6 months), or from 0 days to about a year, or more.

Electroporation vessels containing frozen electrocompetent cells can be stored and maintained at appropriate temperatures, and can also be shipped or otherwise transported from the site of freezing to the point of use. Electroporation vessels containing frozen electrocompetent cells can then be stored at the point of use for convenient access in performing subsequent electroporation steps using the electroporation vessel containing frozen cells.

C. Electroporation

The electroporation vessel containing frozen electrocompetent cells can be thawed, have added thereto a nucleic acid molecule, and be subjected to an electroporation procedure. In some embodiments, the cells are thawed prior to addition of the nucleic acid molecule. In other embodiments, the cells can be completely frozen, or partially thawed upon addition of the nucleic acid molecule. In some embodiments, thawing electrocompetent cells involves increasing the temperature of stored frozen cells (e.g., stored in an electroporation cuvette) to a higher temperature (e.g., ambient temperature, 4° C., 10° C., 20° C., 25° C., 30° C., 35° C. or 40° C.). Upon thawing the cell suspension and adding the nucleic acid molecule, electroporation can be performed.

Nucleic acid molecule can be added by use of a pipette inserted into the electroporation vessel cavity, or by addition through a channel in the electroporation vessel configured for addition of reagents into the electroporation vessel cavity, as known in the art and exemplified in U.S. Pat. No. 6,261,815. The nucleic acid molecule can be mixed with the cell suspension by any of a variety of methods known in the art, including drawing liquid in and out of the pipette, or by tapping the electroporation vessel.

The cuvette can then be placed in an electroporator, and electrical impulses, such as direct current square waves, can be applied to electrodes within the cuvette. A variety of electroporation methods and devices are known in the art, and any device that can accommodate the electroporation vessels provided herein and can apply the intended electrical field, can be used in electroporating the cells of the apparatuses and combinations provided herein. In one example, one electrode can be ground while the other is pulsed, or a positive charge can be applied to one electrode and a negative charge to the other. The charge need not be entirely uniform, and can be somewhat deformed. In another example, electric field pulses can be applied, such as pulses of different duration and voltages can be applied, as known in the art and exemplified in U.S. Pat. No. 6,096,020 to Hofmann et al. In another example, high intensity short duration pulses can be applied, as known in the art and exemplified in U.S. Pat. No. 5,186,800 to Dower et al. In another example, electroporation can be performed with a device that contains a current diverter that diverts current whenever an arc condition commences or other low resistance is detected, as known in the art and exemplified in U.S. Pat. No. 6,258,592 to Ragsdale et al. In another example, electroporation can be performed using a device that automatically determines resistance of a sample and add-on resistance, and applies an appropriate electrical field to the sample, as known in the art and exemplified in U.S. Pub. 20030139889 by Ragsdale et al. The temperature of the electroporation can be controlled using an electroporator with a temperature subsystem, as known in the art and exemplified in U.S. Pat. No. 6,150,148 to Nanda et al. An exemplary electroporation procedure includes applying an electric field at 2500V, 200 ohm, 25 μF to DH10BT1 cells.

A variety of electroporator devices can be used in the electroporation methods provided herein, such as devices that can accommodate and electroporate a plurality of cuvettes, as known in the art and exemplified in U.S. Pub. No. 20030129716 to Ragsdale et al, or devices designed with safety features to permit electroporation to begin after closing the electroporator, as known in the art and exemplified in U.S. Pat. No. 6,699,712, to Kaste et al. Electroporator devices are available from any of a number of manufacturers, such as Bio-Rad (Hercules, Calif.); for example, the Bio-Rad Micropulser Electroporator or Gene Pulser III, can be used.

After electroporation, the cells can be removed from the cuvette, either by automated or manual methods, according to the cuvette used. The cells can then be treated in one or more subsequent steps known in the art for stabilizing the cells, growing the cells, and/or selecting for transformed cells. The cells may be transferred to a medium conducive for cell growth and proliferation, which can vary according to the cell used, as will be known to one skilled in the art. The conducive medium contains one or more components, such as an antibiotic, to assist in selection of transformed cells.

D. Combinations and Kits

Also provided herein are combinations and kits that include frozen electrocompetent cells in an electroporation vessel (e.g., an electroporation cuvette). A combination can also include sterile nutritional media in a separate container. A combination can also contain one or more selection compounds, such as antibiotics, where the selection compounds can be pre-mixed in the nutritional media or separately included. In some embodiments a combination includes one or more nucleic acids (e.g., plasmid and/or polymerase chain reaction primer) in a separate container. In some embodiments a combination includes one or more cloning enzymes (e.g., nucleic acid polymerase, nucleic acid ligase, nucleic acid topoisomerase, uracil DNA glycosylase, protease, phosphatase, ribonuclease, restriction endonuclease, exonuclease and/or ribonuclease inhibitor) in a separate container. A combination can contain a liquid dispenser, such as a disposable pipette, for dispensing a discrete amount of liquid into the cells, for example, a discrete amount of a nucleic acid molecule-containing solution. A combination also can include any other compound, composition or liquid to be added to the electrocompetent cells prior to electroporation.

Kits are packaged combinations that optionally include other reagents or devices. A kit may include literature describing the properties of the cells (e.g., the genotype of the electrocompetent cells). In addition, the kit may contain instructions indicating how the materials within the kit are employed in performing electroporation. Instructions may include a tangible expression describing the reagent concentration or at least one assay method parameter, such as the relative amounts of reagent and sample to be admixed, maintenance time periods for reagent/sample admixtures, temperature, buffer conditions, electroporation settings, and other parameters. The instructions can include directions specifically oriented to the steps, reactants and quantities to be added to the electrocompetent cells according to the aliquot volume of the electrocompetent cells within the electroporation vessel. The kit can include one or more containers capable of holding within fixed limits a composition or buffer solution used in the methods provided herein. A kit also can include substrates, supports or containers for performing electroporation methods, including vials or tubes, or fluid transfer devices such as pipettes. A kit optionally also includes an electroporator. In another example, a kit can contain a tube or flask for growth of the transformed cells. A kit may include literature describing the properties of the bacterial host (e.g., its genotype) and/or instructions regarding its use for transformation.

Kits and combinations also can include a plurality of electroporation cuvettes containing frozen electrocompetent cells. In one embodiment, all electroporation cuvettes contain the same volume of frozen electrocompetent cells. In another embodiment, each cuvette contains a defined volume of frozen electrocompetent cells. A variety of aliquot volumes can be used, as provided herein elsewhere.

The packaging material used in the combination or kit can be one or more physical structures used to house the contents of the combination or kit, and can be constructed by well known methods, to provide a sterile and/or contaminant-free environment. The packaging material can have a label that indicates the components of the combination or kit. The packaging material can also include or contain one or more articles for maintaining the electrocompetent cells in a frozen state, such as a temperature lower than freezing. For example, the packaging material can include dry ice, or other article known in the art for maintaining packaged goods at low temperatures such as the temperatures provided herein for storage of the electroporation vessels containing frozen electrocompetent cells therein.

The following examples are included for illustrative purposes only and are not intended to limit the scope of the invention.

### ILLUSTRATIVE EXAMPLES

### Example 1

### Frozen Cells in Cuvettes

Electrocompetent DH10B E. coli cells and DH10BT1 E. coli cells (Invitrogen, Carlsbad Calif.) were aliquoted into 0.1 cm gap cuvettes. Cells and cuvettes were kept at approximately 4° C. for the aliquoting procedure. The cuvettes were chilled on ice during the process. 25 μl of electrocompetent cells were aliquoted into the cuvettes. Optionally, foil seals were applied to the top of cuvettes to provide an airtight, sterile seal. Plastic snap-caps were placed on top. Cuvettes containing cells were rapidly frozen to −80° C., placed in freezer boxes, and stored at −80° C.

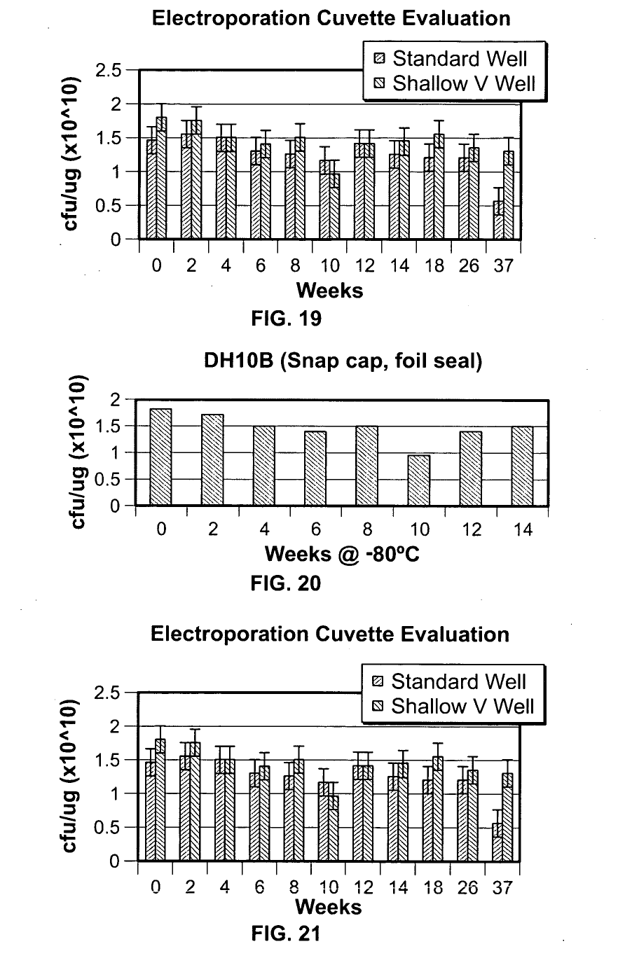

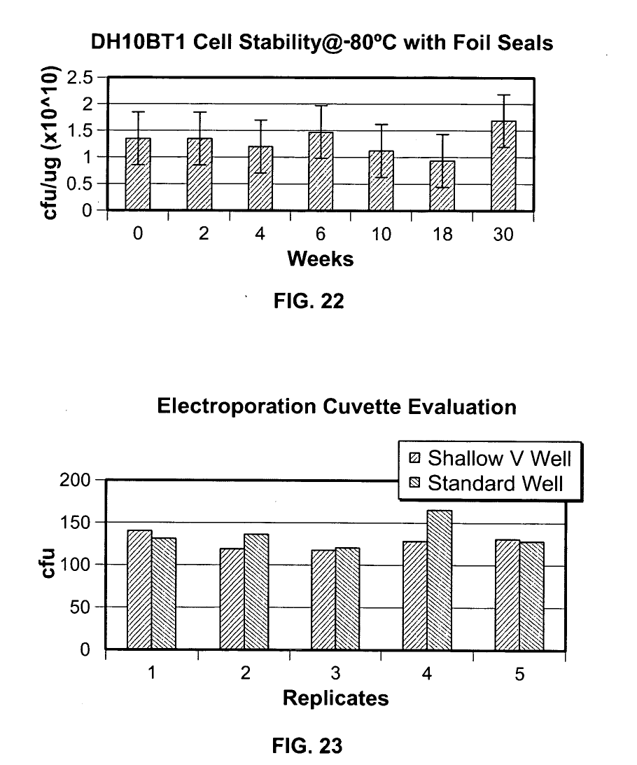

Electroporation cuvettes/cells were placed on ice to thaw after a period of time in storage. Supercoiled pUC 19 DNA was added to the thawed cells, and electroporated in a BTX ECM630 electroporator at 2000 V, 25 μFarads, and 200 ohms. 900 μl SOC media then was added to the cuvette and the mixture transferred to a test tube. After a one hour incubation at 37° C., cells were plated on appropriate media and antibiotic. The charts depicted in FIGS. 19, 20, and 21 show the number of transformants obtained as colony-forming units per microgram of DNA for DH10B cells, and the chart depicted in FIG. 22 shows the number of transformants obtained as colony-forming units per microgram of DNA for DH10BT1 cells.

### Example 2

### Electroporation Cuvettes

Electrocompetent DH 10B E. coli cells (Invitrogen, Carlsbad Calif.) cells were placed into two different types of electroporation cuvettes with different well configurations, on ice. Supercoiled DNA (pUC 19) was added to the cells, and electroporated in a BTX ECM630 electroporator at 2000 V, 25 μFarads, and 200 ohms. 900 μl SOC media then was added to the cuvette and the mixture transferred to a test tube. After a one hour incubation at 37° C., cells were plated on appropriate media and antibiotic. The chart depicted in FIG. 23 shows the number of transformants obtained as colony-forming units per microgram of DNA.

A number of embodiments of the invention have been described. Nevertheless, it will be understood that various modifications may be made without departing from the spirit and scope of the invention. Accordingly, other embodiments are within the scope of the following claims.

1. A method of preparing electrocompetent cells for electroporation, comprising:
adding electrocompetent cells to an electroporation vessel, the vessel comprising one or more electrodes, an upper cavity, and an electroporation well in communication with the upper cavity, wherein the electrocompetent cells are added to the electroporation well; and
freezing the electrocompetent cells in the electroporation well.

2. The method of claim 1, further comprising adding a cover over the cavity, the cover.

3. The method of claim 2, further comprising packaging the electroporation vessel for shipment in an arrangement configured to maintain the cells in a substantially frozen state.

4. The method of claim 2, wherein the cover comprises a tab extending in a generally upward direction.

5. The method of claim 1, wherein the cells are frozen at a temperature of about −90° C.

6. The method of claim 1, wherein the electroporation well in which the electrocompetent cells are added comprises a pair of opposing walls that are downwardly angled toward one another in a gap space between two electrode surfaces.

7. The method of claim 6, wherein the electroporation well in which the electrocompetent cells are added comprises a substantially V-shaped portion in the gap space.

8. The method claim 6, the electroporation well being configured to accommodate a liquid volume of about 35 μL to about 220 μL.

9. The method claim 1, wherein a portion of the cavity being disposed above the electrodes is configured to accommodate a liquid volume of about 700 μL to about 1,100 μL.

10. A method for electroporating cells, comprising:
thawing frozen electrocompetent cells while the cells are in an electroporation vessel having one or more electrodes;
electroporating the electrocompetent cells.

11. The method of claim 10, wherein at least a portion of the frozen electrocompetent cells are thawed while in contact with the one or more electrodes of the electroporation vessel.

12. The method of claim 10, wherein the frozen electrocompetent cells are thawed while being disposed in an electroporation well of the electroporation vessel, the electroporation well comprising a pair of opposing walls that are downwardly angled toward one another in a gap space between two electrode surfaces.

13. The method of claim 12, wherein the electroporation well in which the electrocompetent cells are disposed comprises a substantially V-shaped portion in the gap space.

14. The method of claim 10, wherein the electrocompetent cells are electroporated with one or more nucleic acid molecules.

15. The method of claim 14, wherein electroporating the electrocompetent cells causes at least a portion of the electrocompetent cells to become transformed cells.

16. The method of claim 14, further comprising:
removing the electroporated cells from the electroporation vessel, and
transferring the electroporated cells to a cell proliferation medium.

17. The method of claim 10, wherein the electroporation vessel comprises a substantially quadratic body portion with rounded corners to fit within an electroporator device to applies the electric field.

18. The method of claim 10, wherein the electric field is applied by accessing the bottom surfaces of the one or more electrode and electrically contacting the bottom surfaces of the one or more electrodes.

19. The method of claim 10, further comprising covering an opening of the electroporation vessel with a cap member when electroporating the electrocompetent cells.

20. The method of claim 19, wherein the cap member comprises a tab extending in a generally upward direction.
